(19) United States
(12) Patent Application Publication
(10) Pub. No.: US 2013/0109107 A1
(43) Pub. Date: May 2, 2013

(54) DIAGNOSIS AND TREATMENT OF AUTOIMMUNE DISEASE

(71) Applicants: John C. Hutton, Denver, CO (US); Janet M. Wenzlau, Greenwich Village, CO (US); Howard W. Davidson, Denver, CO (US); Thomas Gardner, Loveland, CO (US)

(72) Inventors: John C. Hutton, Denver, CO (US); Janet M. Wenzlau, Greenwich Village, CO (US); Howard W. Davidson, Denver, CO (US); Thomas Gardner, Loveland, CO (US)

(73) Assignee: The Regents of the University of Colorado

(21) Appl. No.: 13/698,226

(22) PCT Filed: May 25, 2011
(86) PCT No.: PCT/US11/37944

§ 371 Date: Dec. 19, 2012

Related U.S. Application Data
(60) Provisional application No. 61/347,915, filed on May 25, 2010.

(51) Int. Cl. G01N 33/68

(52) U.S. Cl. CPC G01N 33/6854
USPC 436/501; 530/391.1; 530/391.3

(57) ABSTRACT

The detection of parietal cell autoimmune antibodies comprising an ATP4A D3.2 subdomain binding site can diagnose autoimmune body gastritis and/or pernicious anemia with extraordinary sensitivity and specificity that is far superior to existing commercial assays. Further, the assay has diagnostic applications for use in diagnosing type 1 diabetes, thyroiditis and Addison's disease. As pernicious anemia is typically a disease of the elderly, detection of parietal cell antibodies may precede clinical disease by many years if not decades, thereby allowing the initiation of therapeutic interventions such as vitamin B12 administration to prevent the development of pernicious anemia or immunologic interventions to prevent type 1 diabetes and its complications.

### FIELD OF INVENTION

This invention relates to the diagnosis and treatment of autoimmune disease. Specifically, an improved immunodiagnostic method is disclosed that improves autoantibody detection sensitivity such that early diagnosis may be obtained. For example, the method utilizes an epitope within the ATP4A chain region D3.2. Consequently, ATP4A autoimmunity may reflect suseptibility to a panel of autoimmune diseases including but not limited to diabetes, thyroid disease, rheumatoid arthritis, autoimmune body gastritis, and/or pernicious anemia.

### BACKGROUND

Even before an autoimmune basis for type 1 diabetes (T1D) was confirmed, a significant incidence of parietal cell autoantibodies (PCAs) in insulin-dependent diabetic patients was noted. Bottazzo et al., “Islet-cell antibodies in diabetes mellitus with autoimmune polyendocrine deficiencies” Lancet 2: 1279-1283 (1974); and Ungar et al., “HLA-DR patterns in pernicious anemia” British Medical Journal (Clip Res Ed) 282:768-770 (1981). The presence of circulating PCAs was believed to be indicative of atrophic body gastritis (ABG), an autoimmune disease which in its chronic form manifests as perniciouos anemia (PA).

PCAs have been shown to bind to both an ˜100 kD α-subunit and an approximate 60-90 kD heavily glycosylated β-sbunit of the ATP4A/B heterodimer. Dar et al., “Characterization of Na,K-ATPase and H,K-ATPase enzymes with glycosylation-deficient beta-subunit variants by voltage-clamp fluorometry in Xenopus oocytes” Biochemistry 47: 4288-4297 (2008). Major epitopes in a human β-subunit may be dependent on a full complement of N-linked glycans for immunoreactivity. Goldkorn et al., “Gastric parietal cell antigens of 60-90, 92, and 100-120 kDa associated with autoimmune gastritis and pernicious anemia. Role of N-glycans in the structure and antigenicity of the 60-90-kDa component” Journal of Biological Chemistry 264:18768-18774 (1989); and Stewart et al., “Species-specific distribution of alpha-galactosyl epitopes on the gastric H/K ATPase beta-subunit: relevance to the binding of human anti-parietal cell autoantibodies” Glycobiology 9:601-606 (1999).

Humoral epitopes for the human α and β subunits have been identified by immunization of mice with overlapping human peptides and selection of those that induce inflammatory infiltration specifically in the gastric mucosa in mice, followed by loss of acid-secreting parietal cells and the appearance of circulating autoantibodies directed to ATP4. D'Elios et al., “Helicobacter pylori, T cells and cytokines: the “dangerous liaisons””FEMS Immunology and Medical Microbiology 44:113-119 (2005).

The potassium/hydrogen ion transporter (ATP4 or H⁺/K⁺ ATPase) is an enzyme located principally in the parietal cell of the stomach that may be responsible for the acidification of the gastric juice. Acidification serves as a barrier to the entry of harmful microorganisms and toxins into the gastrointestinal tract and facilitates the initial digestion of proteins by the enzyme pepsin. ATP4 can be a target of antibodies and T-lymphocytes in diseases, including but not limited to, autoimmune body gastritis (ABG), a condition that leads to dysfunction and destruction of the parietal cell and malabsorption of dietary vitamin B12, the serious clinical condition termed pernicious anemia which if untreated leads to irreversible neurodegeneration.

While current methods have identified the relationships between autoimmune antibodies and autoimmune diseases, they have not become sufficiently sensitive to identify early phase patients such that long-term complications of these diseases can be avoided. What is needed in the art is a broad-based diagnostic platform that has vastly improved sensitivity such that a developing autoimmune disease may be diagnosed before the appearance of an overt symptomology pattern.

### SUMMARY OF THE INVENTION

This invention relates to the diagnosis and treatment of autoimmune disease. Specifically, an improved immunodiagnostic method is disclosed that improves autoantibody detection sensitivity such that early diagnosis may be obtained. For example, the method utilizes an epitope within the ATP4A chain region D3.2. Consequently, ATP4A autoimmunity may reflect susceptibility to a panel of autoimmune diseases including but not limited to diabetes, thyroid disease, rheumatoid arthritis, autoimmune body gastritis, and/or pernicious anemia.

In one embodiment, the present invention contemplates an assay using a molecularly optimized ATP4A probe that exhibits exquisite sensitivity and specificity. In one embodiment, the assay comprises a radioimmunoassay capable of detecting type 1 diabetes (T1D) antigens. Although it is not necessary to understand the mechanism of an invention, it is believed that there may be an association of PA with some autoimmune diseases (i.e., for example, T1D). In one embodiment, the assay determines the prevalence of PCAs in newly diagnosed T1D individuals. In some embodiments, the assay associates PCA prevalence with T1D autoantibodies and/or patient gender.

In one embodiment, the present invention contemplates a complex comprising an autoimmune antibody having an ATP4 D3.2 subdomain binding site and an ATP4 D3.2 subdomain antigen attached to the ATP4 D3.2 subdomain binding site. In one embodiment, the autoimmune antibody is a stomach parietal cell antibody. In one embodiment, the autoimmune antibody is a pancreatic islet cell antibody. In one embodiment, the autoimmune antibody is a thyroid antibody. In one embodiment, the autoimmune antibody is a rheumatoid arthritis antibody. In one embodiment, the antigen comprises a label. In one embodiment, the label is a radioactive label. In one embodiment, the radioactive label is ³⁵S.

In one embodiment, the antigen comprises an amino acid sequence. In one embodiment, the amino acid sequence comprises at least 215 amino acids.

In one embodiment, the present invention contemplates a method, comprising: a) providing; i) a biological sample comprising an autoimmune antibody, wherein the autoimmune antibody comprises an ATP4 D3.2 subdomain binding site; ii) an ATP4 D3.2 subdomain antigen having specific affinity for the ATP4 D3.2 subdomain binding site; and b) contacting the biological sample with the antigen under conditions such that the autoimmune antibody is identified. In one embodiment, the conditions comprise immunoprecipitation of the autoimmune antibody. In one embodiment, the conditions comprise identifying the autoimmune antibody with at least at a 95% sensitivity. In one embodiment, the conditions comprise identifying the autoimmune antibody at least at a 96%% sensitivity. In one embodiment, the conditions comprise identifying the autoimmune antibody at least at 97% sensitivity. In one embodiment, the biological sample is a blood sample. In one embodiment, the blood sample is selected from the group consisting of a whole blood sample, a plasma sample, or a serum sample. In one embodiment, the biological sample is a stomach sample. In one embodiment, the biological sample is a pancreas sample. In one embodiment, the biological sample is a thyroid sample. In one embodiment, the sample is a saliva sample. Although it is not necessary to understand the mechanism of an invention, it is possible that the ATP4 D3.2 antigen may not be derived from either the pancreas or thyroid, even though antibodies comprising an ATP4 D3.2 subdomain binding site are associated with autoimmune diseases involving these tissues.

In one embodiment, the present invention contemplates a method, comprising: a) providing; i) a patient exhibiting symptoms of an autoimmune disease; ii) a labeled ATP4A D3.2 subdomain antigen; and b) obtaining a biological sample from the patient; and c) using the antigen to identify an autoimmune antibody in the biological sample. In one embodiment, the autoimmune antibody comprises an ATP4A D3.2 subdomain binding site. In one embodiment, the autoimmune antibody is an autoimmune body gastritis antibody. In one embodiment, the autoimmune antibody is a type 1 diabetes antibody. In one embodiment, the autoimmune antibody is a pernicious anemia antibody. In one embodiment, the autoimmune antibody is a thyroiditis antibody. In one embodiment, the autoimmune antibody is an Addison's disease antibody. In one embodiment, the autoimmune antibody is a rheumatoid arthritis antibody.

In one embodiment, the present invention contemplates a method, comprising: a) providing; i) a patient exhibiting symptoms of an autoimmune disease; ii) a labeled ATP4A D3.2 subdomain antigen; and b) obtaining a biological sample from the patient; and c) using the antigen to diagnose the autoimmune disease of the patient. In one embodiment, the diagnosis is autoimmune body gastritis. In one embodiment, the diagnosis is type 1 diabetes. In one embodiment, the diagnosis is pernicious anemia. In one embodiment, the diagnosis is thyroiditis. In one embodiment, the diagnosis is Addison's disease. In one embodiment, the diagnosis is rheumatoid arthritis.

In one embodiment, the present invention contemplates a method, comprising: a) providing; i) a patient at risk of developing symptoms of an autoimmune disease; ii) a labeled ATP4A D3.2 subdomain antigen; and b) obtaining a biological sample from the patient; and c) using the antibody to identify an autoimmune antibody, wherein the autoimmune antibody comprises an ATP4A D3.2 subdomain binding site. In one embodiment, the method further comprises after step (c), administering a therapeutic intervention. In one embodiment, the therapeutic intervention comprises vitamin B12. In one embodiment, the therapeutic intervention comprises an anticancer agent. In one embodiment, the therapeutic intervention comprises an antidiabetic agent. In one embodiment, the therapeutic intervention comprises an antigastrin agent. In one embodiment, the therapeutic intervention comprises an anti-inflammatory agent. In one embodiment, the conditions comprise immunoprecipitation of the autoimmune antibody. In one embodiment, the conditions comprise identifying the autoimmune antibody at least at 95% sensitivity. In one embodiment, the conditions comprise identifying the autoimmune antibody at least at 96% sensitivity. In one embodiment, the conditions comprise identifying the autoimmune antibody at least at 97% sensitivity. In one embodiment, the biological sample is a blood sample. In one embodiment, the blood sample is selected from the group consisting of a whole blood sample, a plasma sample, or a serum sample. In one embodiment, the biological sample is a stomach sample. In one embodiment, the biological sample is a pancreas sample. In one embodiment, the biological sample is a thyroid sample. In one embodiment, the sample a saliva sample.

In one embodiment, the present invention contemplates a kit comprising: a) a first container comprising a labeled ATP4A D3.2 subdomain antigen; b) a second container comprising buffers and reagents compatible with the antigen; and c) instructions describing the use of the first and second containers to identify an autoimmune antibody from a biological sample.

In one embodiment, the present invention contemplates a method comprising; a) providing; i) a patient suspected of comprising an ATP4A autoantibody; and ii) a biological sample derived from the patient; iii) a labeled ATP4A antigen capable of binding to the ATP4A autoantibody; b) contacting the labeled ATP4A antigen with the biological sample; and c) determining the ATP4A autoantibody level. In one embodiment, the ATP4A autoantibody comprises an ATP4A D3.2 subdomain. In one embodiment, the patient is diagnosed with type 1 diabetes within the last six months. In one embodiment, the patient is diagnosed as at risk for type 1 diabetes. In one embodiment, the patient is diagnosed with autoimmune body gastritis (ABG). In one embodiment, the detection of the ATP4A autoantibody diagnoses ABG. In one embodiment, the ATP4A autoantibody level increases with the age of the patient at diagnosis. In one embodiment, the method further comprises determining an ATP4A autoantibody index. In one embodiment, the ATP4 autoantibody index is gender biased. In one embodiment, the gender bias is female. In one embodiment, the biological sample comprises a saliva sample. In one embodiment, the biological sample comprises a blood sample. In one embodiment, the blood sample is selected from the group including but not limited to a whole blood sample, a serum sample, and/or a plasma sample. In one embodiment, the biological sample is a tissue sample.

### DEFINITIONS

The term “autoimmune antibody” as used herein refers to any antibody having a specific affinity for a naturally occurring biological compound (i.e., for example, a protein, peptide, carbohydrate, lipid or nucleic acid). In some cases, the binding of the autoimmune antibody and the naturally occurring biological compound may result in a disease (i.e., for example, an autoimmune disorder).

The term “ATP4 protein” or “ATP4 enzyme” as used herein refers to any phosphatase enzyme comprising an alpha subunit (ATP4A) and a beta subunit (ATP4B). For example, an ATP4A subunit may comprise amino acid residue positions 1-1035. Within this subunit, the D3 domain comprises amino acid residue positions 350-783. Within the D3 domain, the D3.1 subdomain comprises amino acid residue positions 350-393 (whose secondary structure is associated with amino acid residue positions 597-783) and the D3.2 subdomain comprises amino acid residue positions 394-596.

The term “ATP4 D3.2 subdomain binding site” as used herein, refers to any site within an antibody (i.e., for example, an autoimmune antibody) having specific affinity for an ATP4A D3.2 antigen. For example, a binding site (i.e., for example, an antigen binding site) within the ATP4A D3.2 subdomain may comprise amino acids 394 to 596 of an H⁺/K⁺ ATPase enzyme.

The term “ATP4Q D3.2 subdomain antigen” as used herein, refers to any amino acid sequence derived from an ATP4A D3.2 subdomain or portion thereof.

The term “immunoprecipitation” as used herein, refers to any precipitation of a complex of an antibody and its specific antigen. Usually, such a complex may be initiated by the addition of a protein that binds immunoglobulin including, but not limited to, Protein A on an agarose solid support.

The term “sensitivity” as used herein, means the frequency with which a laboratory method correctly detects a known condition such as a disease state. Sensitivity is calculated as the number of true positive results divided by the sum of the true positives and false negatives. For example, an autoantibody value can be selected such that the sensitivity of detecting an autoantibody is at least about 60%, and can be, for example, at least about 65%, 70%, 75%, 80%, 85%, 90%, 95%, 96%, 97%, 98%, 99%, or 100%.

The term “specificity” as used herein, means the accuracy with which a laboratory method correctly excludes individuals who do not have a known condition such as a disease state, and relates to the frequency of false positive results. Specificity is calculated as the number of true negative results divided by the sum of the true negatives and false positives. For example, an autoantibody cut-off value can be selected such that the specificity is in the range of 30-100%, for example, at least about 30%, 50%, 75%, 80%, 90%, 95%, 98%, 99%, or 100%. This means that a positive signal will be obtained from at least about 70%, 50%, 25%, 20%, 10%, 5%, 2%, 1%, or 0%, respectively of subjects who do not have the specific known condition.

The term “anticancer agent” as used herein, refers to any compound having known effectiveness to reduce symptoms of cancer. For example, reduction in symptoms may include, but are limited to, diminished size of a tumor, reduced number of tumors, or reduced lymphocyte levels. An anticancer agent may include but is not limited to, taxol, actinomycin-D, cis-platinum, BiCNU, adriamycin, doxorubicin, fluorouracil, methotrexate, thioguanine, or vincristine.

The term “antidiabetic agent” as used herein, refers to any compound having known effectiveness to reduce symptoms of diabetes. For example, reduction in symptoms may include but are not limited to, reduced urinary glucose or reduced plasma glucose. An antidiabetic agent may include but is not limited to, insulin, metformin or a siulphonylurea.

The term “anti-gastrin agent” as used herein, refers to any compound having known effectiveness to reduce gastrointestinal secretion of gastrin. For example, an antigastrin agent may include, but is not limited to, an H1 receptor blocker.

The term “anti-inflammatory agent” as used herein, refers to any compound having known effectiveness to reduce symptoms of inflammation. For example, reduction in symptoms may include, but are not limited to, reduced swelling, redness and/or local lymphocyte levels. For example, an anti-inflammatory agent may include, but is not limited to, aspirin, acetominophen, ibuprofen, cortiocosterone, or cortisol.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “symptom”, as used herein, refers to any subjective or objective evidence of disease or physical disturbance. For example, subjective evidence is usually based upon patient self-reporting, medical testing, and/or observations by qualified medical personnel and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease”, as used herein, refers to any impairment of the normal state of the living animal or plant body or one of its parts that interrupts or modifies the performance of the vital functions. Typically it is manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, an emulsion with and the like.

The term “drug”, “agent” or “compound” as used herein, refers to any pharmacologically active substance capable of being administered which achieves a desired effect. Drugs or compounds can be synthetic or naturally occurring, non-peptide, proteins or peptides, oligonucleotides, or nucleotides (DNA and/or RNA), polysaccharides or sugars.

The term “administered” or “administering”, as used herein, refers to any method of providing a composition to a patient such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “patient”, as used herein, is a human or animal and need not be hospitalized. For example, out-patients, and persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “biocompatible”, as used herein, refers to any material that does not elicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

“Nucleic acid sequence” and “nucleotide sequence” as used herein refers to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide (or modified protein or peptide) means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabelled A) in a reaction containing labeled “A” and the antibody will reduce the amount of labeled A bound to the antibody.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “immunologically active” defines the capability of a natural, recombinant modified or synthetic peptide, or any oligopeptide or nucleic acid thereof, to induce a specific immune response in appropriate animals or cells and/or to bind with specific antibodies.

The term “antigenic determinant” as used herein refers to that portion of a molecule that is recognized by a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “immunogen,” “antigen,” “immunogenic” and “antigenic” refer to any substance capable of generating antibodies when introduced into an animal. By definition, an immunogen must contain at least one epitope (the specific biochemical unit capable of causing an immune response), and generally contains many more. Proteins are most frequently used as immunogens. However, lipid and nucleic acid moieties may also act as immunogens, either individually or as a protein complex. The latter complexes are often useful when smaller molecules with few epitopes do not stimulate a satisfactory immune response by themselves.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence that is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed to a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which inhibit hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an anti-parallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments of SEQ ID NO:2 are employed in hybridization reactions under stringent conditions the hybridization of fragments of SEQ ID NO:2 which contain unique sequences (i.e., regions which are either non-homologous to or which contain less than about 50% homology or complementarity with SEQ ID NOs:2) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA (or cDNA) to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element, in the coding region or intervening sequences. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operationally linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest and what level of gene expression is desired.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning. A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligoribonucleotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb (or more) on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters, enhancers or suppressors which control or influence the transcription of the gene. The 3′ flanking region may contain sequences which direct the termination of transcription, stability of a mRNA, posttranscriptional cleavage and polyadenylation.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The term “binding” as used herein, refers to any interaction between an infection control composition and a surface. Such as surface is defined as a “binding surface”. Binding may be reversible or irreversible. Such binding may be, but is not limited to, non-covalent binding, covalent bonding, ionic bonding, Van de Waals forces or friction, and the like. An infection control composition is bound to a surface if it is impregnated, incorporated, coated, in suspension with, in solution with, mixed with, etc.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 illustrates a conventional radiochemical/immunoprecipitation assay to detect autoimmune antibodies.

FIG. 2 presents one embodiment of the subdomain structure of a I-1⁺/K⁺ ATPase.

FIG. 2A: A schematic representation of H⁺/K⁺ ATPase showing subdomains comprising D1, D2, and D3.

FIG. 2B: A colorized modeling of the three-dimensional structure of one embodiment of an ATPase4A D3.1 subdomain and an ATPase4A D3.2 subdomain. D3.1 subdomain: Circled in green (amino acids 1-44; D3.2 subdomain: Circled in red (amino acids 45-247); and a Ser, Gly, Ser, Gly, Ser linker (amino acids 248-C-terminus)

FIG. 3 presents exemplary data showing the iterative testing of various ATP4A region 3.2 subdomains.

FIG. 3A illustrates several embodiments of ATP4A D3.2 variants having either N- or C-terminal end extensions or deletions of approximately 10 amino acids.

FIG. 3B presents exemplary data showing the capability of various ATP4A D3.2 variants of detecting autoantibodies in newly diagnosed T1D patients (NNO patients) relative to the wild type ATP4A D3.2 sequence comprising amino acids 1-125.

FIG. 4 presents exemplary data demonstrating the presence of ATP4A D3.2 epitopes in sera derived from ABG patients whose diagnosis has been confirmed by biopsy.

FIG. 4A plots the sensitivity versus specificity in receiver/operator curves of the ATP4A D3.2 epitope in detecting autoantibodies of ABG patients.

FIG. 4B presents a scatter plot of the data summarized in FIG. 4A.

FIG. 5 presents exemplary data demonstrating the improved sensitivity of the ATP4A D3 radioimmunoprecipitation assay versus a conventional ELISA method to detect ABG autoantibodies.

FIG. 6 presents alignments of various homologous proteins in the region comprising the ATP4A D3.2 subdomain.

FIG. 7 presents exemplary data showing the relative abilities of ATP4A D3.2 homologs to detect T1D autoimmune antibodies.

FIG. 8 presents exemplary data suggesting that ATP4A is expressed in pancreatic islet cells.

FIG. 9 presents a comparison of specific regions between various embodiments of ATP4A homologs.

FIG. 10 presents exemplary data showing the detection of ATP4A D.3 epitopes with diabetes mellitus autoantibodies.

FIG. 10A: Exemplary data showing the detection of ATP4A D.3 epitopes in sera containing T1D autoantibodies.

FIG. 10B: Exemplary data showing the detection of ATP4A D.3 epitopes in T1D negative, autoantibody negative sera. This data also demonstrates that newly diagnosed diabetic patients with known diabetes related antibodies to the antigens IA2, insulin, and GAD65 are more likely to have antibodies to ATP4A D3.2 and that the titers are higher.

FIG. 11 presents exemplary data showing the sensitivity and specificity of detecting diabetes autoimmune antibodies comprising ATP4A D.3 epitopes in newly diagnosed diabetes patients. The diabetes patients are stratified on whether they have diabetes related antibodies. Patients having diabetes-related antibodies are more likely to have ATP4A D3.2 antibodies

FIG. 12 presents exemplary data showing the relationship of autoimmune antibodies comprising an ATP4A D.3 epitope in sera from individuals with various autoimmune diseases. Data is shown with 95% confidence intervals.

FIG. 13 presents exemplary data showing the sensitivity and dynamic range of ATP4A D3 autoantibody assay in sera from ABG patients. Further, the RIA has a dynamic range>10⁴ when combined with dilution.

FIG. 13A: Concordance in 2.5 ul sera samples.

FIG. 13B: Concordance in 0.1 ul sera samples.

FIG. 14 presents exemplary data of the relative reactivity of various ATP4B probes in a radioimmunoassay.

FIG. 15A presents exemplary data showing control and diabetic subjects (numbers in parentheses) that were assayed with a radioimmunoprecipitation assay using either a full length ATP4A molecule as an antigen, or a fragment of the third intracellular domain (D3.2) as an antigen.

FIG. 15B presents exemplary data of a frequency distribution analysis of the data in FIG. 15A showing that the when using a D3.2 antigen, a lower non-specific binding is observed in concert with an enhanced ability to discriminate antibody positive diabetic patients by virtue of a higher signal to noise ratio.

FIG. 16 presents exemplary data comparing autoantibody detection of “gold standard” T1D antigens and ATP4A antigens. Immunoreactivity of sera from 116 newly diagnosed T1D individuals was determined for ATP4A, GAD65, ICA512, MIAA, and ZnT8 antigens. Values are expressed as percentage of patients testing positive for the indicated antigens with respect to age of onset.

FIG. 17 presents exemplary immunoreactivity data demonstrating gender bias of ATP4A autoantigens as compared to “gold standard” T1D autoantigens. Autoantibodies for ATP4A, GAD65, Insulin, IA2 and ZnT8 were measured in 463 newly diagnosed T1D individuals. Autoantibody positive sera was stratified for gender and expressed as a binding index ((mean of sample binding−mean of negative controls)/(mean of positive controls−mean of negative controls)).

FIG. 18 presents an alignment of the D3.2 region in gene family members defining regions of conservation, putative epitopes and outlines 4 domains that are both clustered and predicted to reside on the surface of the molecules. Regions from such homologues were cloned and tested for immunoreactivity in RIAs with sera that had high medium and low reactivity to the ATPase a probe. Reactivity to homologues is predictably the highest with a subset of highly reactive ATPase4A positive samples only with ATPase12A. Regions conserved between ATPase4A and 12A may define conserved epitopes. Family member ATPase1A1, with no immunoreactivity was employed as a partner to conserve structure in chimeric constructs to map epitope domains in ATPase4A. Immunoreactivity appears to be localized to the N-terminal half of ATPase4A D3 containing two regions of amino acids unique to ATPase4A (see blue boxes).

FIG. 19 presents chimeric embodiments of ATP4A and probable interactions within a cell membrane (i.e., for example, a parietal cell membrane).

FIG. 20 presents exemplary data of relative immunoreactivity of various ATP4A homologs.

FIG. 21 presents exemplary data showing that ATP4A autoantibodies in type 1 diabetic subjects increase in accordance with the patient's age at diagnosis. Sera from 135 T1D subjects was assayed by radioimmunoprecipitation for IA2 (ICA512) autoantibodies and ATPase4A antibodies to determine prevalence with duration of disease. Diabetes-specific (ICA512) antibodies decreased with age while ATP4A D3 antibodies increased with age. In this example, the prevalence of ATP4A D3 antibodies is higher in diabetics (16%) than in age matched controls (8%).

FIG. 22 presents exemplary data showing that “gold standard” T1D antibodies in type 1 diabetic subjects do not increase over time (e.g., ZnT8, IA2, and/or GAD65).

FIG. 23 presents exemplary data demonstrating the presence of ATP4A autoantibody in young subjects (e.g., between approximately 1-15 years) at risk of developing T1D.

FIG. 24 presents various embodiments contemplated by the present invention of ATP4 constructs.

### DETAILED DESCRIPTION OF THE INVENTION

This invention relates to the diagnosis and treatment of autoimmune disease. Specifically, an improved immunodiagnostic method is disclosed that improves autoantibody detection sensitivity such that early diagnosis may be obtained. For example, the method utilizes an epitope within the ATP4A chain region D3.2. Consequently, ATP4A autoimmunity may reflect susceptibility to a panel of autoimmune diseases including but not limited to diabetes, thyroid disease, rheumatoid arthritis, autoimmune body gastritis, and/or pernicious anemia.

Autoimmune body gastritis (ABG) and pernicious anemia (PA) are prototypical, organ-specific autoimmune diseases whose prevalence in the general population are between approximately 2 and 0.15-1%, respectively. The incidence of these diseases increases with age and is frequently associated with other autoimmune disorders including but not limited to type 1 diabetes (T1D). Early diagnosis of ABG and/or PA serves to either i) prevent and/or ii) provide immediate treatment before consequences of chronic disease become irreversible. Parietal cell autoantibody (PCA) detection via ELISA is currently the most widely used biomarker of disease, wherein a diagnosis must be confirmed by subsequent immunohistochemistry via biopsy.

Although vitamin B12 replacement therapy has been promoted to treat PA, the disease has continued to be associated with numerous devastating complications including, but not limited to, irreversible neuropathy and/or gastric adenocarcinoma. Gastric H⁺/K⁺-ATP4 (ATP4) has been suggested to an antigen recognized by PCAs. ATP4 has been reported as an α/β-heterodimeric integral membrane protein that may be responsible for gastric acidification by parietal cell secretion of hydrogen ions in exchange for K⁺ ions. Prinz et al., “Acid secretion and the H,K ATPase of stomach” Journal of Biological Medicine 65: 577-596 (1992); Varis et al., “Serum pepsinogen I and serum gastrin in the screening of atrophic pangastritis with high risk of gastric cancer” Scandanavian Journal of Gastroenterolology Suppl. 186: 117-123 (1991); De Block et al., “Helicobacter pylori, parietal cell antibodies and autoimmune gastropathy in type 1 diabetes mellitus” Aliment Pharmacology Therapy 16:281-289 (2002); and Annibale et al., “Antral gastrin cell hyperfunction in children. A functional and immunocytochemical report” Gastroenterology 101:1547-1551 (1991).

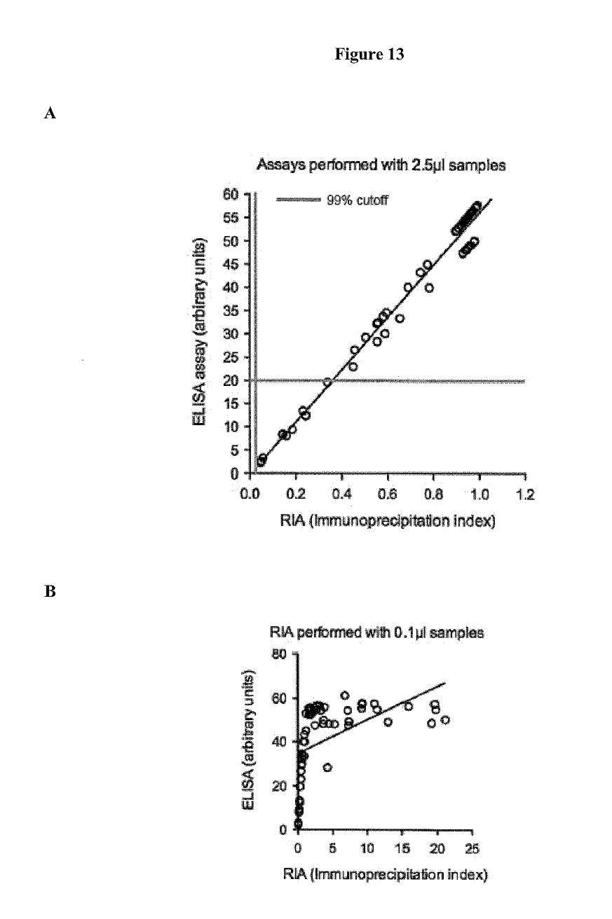

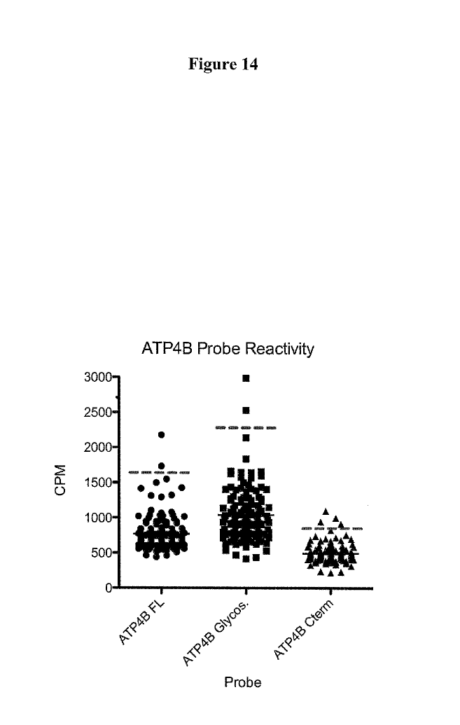

In one embodiment, the present invention contemplates a diagnostic method comprising detecting parietal cell autoantibodies. In one embodiment, the method further comprises an autoantibody comprising affinity to at least a portion of an ATP4A chain region D.3 (i.e., for example, an ATP4A/D.3 antigen). Although it is not necessary to understand the mechanism of an invention, it is believed that this ATP4A D.3 antigen/autoantibody affinity provides an improved assay over those currently available, as being more sensitive, highly specific, faster and more precise. In one embodiment, the method further comprises radioimmunoprecipitation. For example, the presently disclosed method is compared with a commercial PCA assay on patients with ABG. See, FIGS. 13A and 13B. The data show a strong correlation between the presently disclosed method and the commercial PCA assay. These data verify that the presently disclosed assay is a reliable diagnostic for PCA.

TABLE 1
Concordance Between ATP4A D3 RIA
And Conventional ELISA In ABG

| | − | RIA | + |
|---|---|---|---|
| + | 7 | | 34 |
| ELISA | | | |
| − | 18 | | 33 |

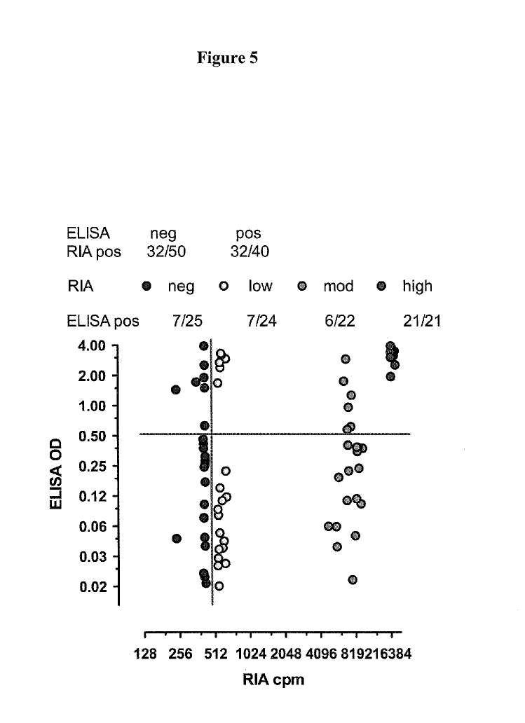

One advantage of the presently disclosed assay is an improved sensitivity, as demonstrated by the cutoff value. See, FIG. 5. RIAs were performed on plasma from patients who had undergone optic biopsy to confirm clinical diagnosis. Plasma from controls were assayed with 2.5 ABG patients with 2.5 μl initially and repeated at 0.1 μl if index exceeded 0.8. Compare, FIG. 13A and FIG. 13B, respectively. The dilution was factored into the final calculation. Given the specificity and sensitivity of the RIA in ABG patients, it is surprising that the same antibodies are also present in non-ABG patients exhibiting symptoms of other autoimmune diseases (i.e., for example, diabetes). Although it is not necessary to understand the mechanism of an invention, it is believed that this observation may be detecting an underlying defect in central immune tolerance that can be triggered by an environmental factor, in this case possibly Helicobacter pylori infection.

### I. Current Methods For Diagnosing Autoimmune Diseases

A. Autoimmune Body Gastritis

It is believed that autoimmune body gastritis (ABG) may be associated with gastric H⁺, K⁺-adenosine triphosphatase (ATPase) specific autoantibodies (H⁺/K⁺ Ab) as determined from partially purified preparations of pig stomach that is enriched in H⁺/K⁺ ATPase as defined by catalytic activity. Until the present invention, the specific autoantigen (i.e., for example, a molecularly defined epitope) that triggers this disease was unknown. In some embodiments, the present invention contemplates an improvement of ABG diagnosis assays by detecting the ATP4A D.3 domain that comprises such autoantigens.

Nonetheless, previous studies have reported that autoantibodies are produced during autoimmune gastritis in neonatally thymectomized mice and investigated whether a native H⁺/K⁺-ATPase antigen preparation can induce autoimmune disease in mice. Claeys et al., “Neonatal Injection of Native Proton Pump Antigens Induces Autoimmune Gastritis in Mice” Gastroenterology 113:1136-1145 (1997). This method characterized the autoantibodies by immunoprecipitation of a functional full length H⁺/K⁺-ATPase expressed in Xenopus oocytes. Conformational autoantibodies recognizing both H⁺/K⁺-ATPase A and beta b appeared simultaneously with the gastric lesions 1 month after thymectomy. This protocol, however, was limited to inducing autoimmune gastritis only in neonate mice and not in adult mice.

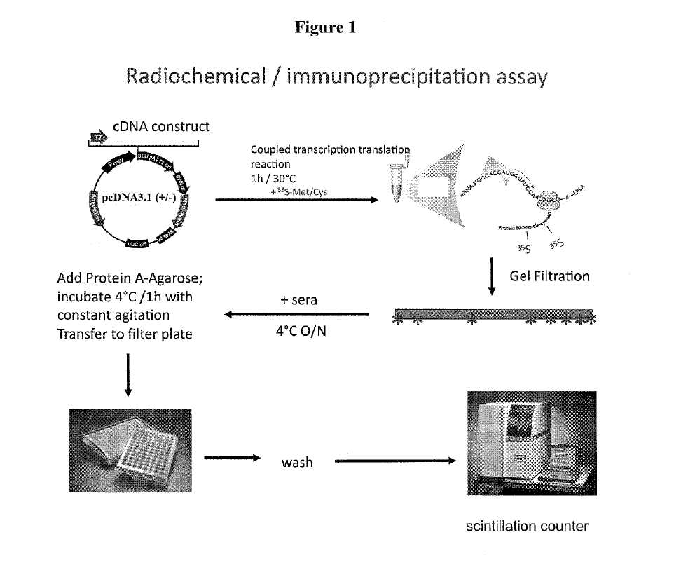

In one embodiment, the present invention contemplates a method to diagnose ABG utilizing a high throughput 96 well plate radiochemical/immunoprecipitation assay that may also be used to measure autoantibodies for insulin, GAD, IA2 and ZnT8 in the diagnosis of diabetes. See, FIG. 1. In one embodiment, a recombinant full length enzyme is produced using a plasmid construct labeled by radioactive ³⁵S-methionine or ³⁵S-cysteine incorporation. In one embodiment, the radioactive protein is gel filtered and then transferred onto a well plate and incubated with the autoantibody-containing serum sample. In one embodiment, the incubated autoantibody-antigen complex is precipitated with Protein A agarose, wherein the precipitated autoantibody/radiolabeled enzyme complexes are quantitated by radioactivity detection using a scintillation counter.

The radioimmunoassay described above has a higher sensitivity than other common assays for ABG comprising, for example, histochemistry wherein autoantibodies are detected using antibodies comprising fluorescent labels. In this assay, biopsied stomach tissue sections are taken and autoantibody binding is detected by immunofluorescence. Histochemical staining in ABG was one of the first examples of detecting autoantibodies associated with a disease. Immunofluorescence techniques were later extended to diabetes (ICA) and provided assays that were sensitive but required operator training and proved subjective and difficult to standardize. Other fluorescence assays may utilize recombinant fusion protein assays based on known luciferase-luciferin reporter techniques including, Guassia (BioLux Gaussia Luciferase Assay Kit (E3300), Reporter Systems, NEB), firefly luciferase, or Renilla luciferase, wherein the data is obtained by measuring light production in a luminometer after addition of appropriate luciferase substrates.

Alternatively, ABG may be identified using an enzyme linked immunoabsorbent assay (ELISA), wherein pig pancreas microsomal protein is bound to a plate and autoantibody binding is detected with an enzyme conjugate comprising a reporter molecule. ELISA assays, like histochemical assays, require a biological source of material and biochemical purification of the antigen (typically from pig stomach). Cross-reaction of autoantibodies with other antigens and Fc receptors can be a problem and it is difficult to standardize the ligand preparation. Sensitivities are typically 80% at 90% specificity.

While it can be argued that ELISA could have a similar sensitivity as MA, because ELISA detects both ATP4A and ATP4B (the other subunit of the enzyme), ELISA confers no advantage over RIA because all ATP4B positive patients are also ATP4A positive. The data presented herein, demonstrate that an radioimmunoprecipitation assay for ATP4B confirms this conclusion. Further, ATP4B assays generally have a very high background, wherein any attempt to combine these two assays would be expected to decrease the sensitivity.

Current ELISA assays directed towards parietal cell autoantibodies (PCAs) and intrinsic factor autoantibodies (IFAs) have been used because of ABG's relationship to cobalamin deficiency. This combination of IFA and PCA testing resulted in a 73% sensitivity for PA diagnosis. Lahner et al., “Reassessment of Intrinsic Factor and Parietal Cell Autoantibodies in Atrophic Gastritis With Respect to Cobalamin Deficiency” Am J Gastroenterol 104:2071-2079 (2000).

One current assay discloses an antibody to an 1-1⁺/K⁺-ATPase chain as a possible gastritis biomarker. The method suggests diagnosing the possible presence of gastritis in a human by evaluating a blood sample for the combination of autoantibodies specific for H⁺/K⁺-ATPase, Helicobacter pylori antibodies, and pepsinogen I concentration. A potential gastritis diagnosis would depend upon an integrated analysis (i.e., for example, by using a custom software program) that evaluates the relative levels of H⁺/K⁺-ATPase antibodies, Helicobacter pylori antibodies, and pepsinogen I concentration. Mardh et al., “Screening Method For Gastritis” U.S. Pat. No. 7,179,609 (herein incorporated by reference); and EP1488238. The overall sensitivity of this technique to detect gastritis using this assay was 88% (211/240) (95% CI 83 92%) with a specificity of 81% (196/243) (95% CI 75 85%).

B. Pernicious Anemia

Currently, most clinical approaches to diagnosing pernicious anemia are dependent upon a three-pronged testing paradigm. First, the presence of ABG may be evaluated based upon the measurement of serological markers, such as increased fasting gastrin and reduced levels of pepsinogen I in combination with a histological confirmation by biopsy sampling of gastric body mucosa. Second, the patient may be evaluated for a deficiency in intrinsic factor (IF) by determining the presence of IF antibodies or stomach parietal cell antibodies (these methods have replaced the Schilling test). Finally, testing may be performed to determine whether a colbalamin deficiency exists in combination with macrocytic anemia.

Pernicious anemia (PA) is believed to be a macrocytic anemia that is caused by vitamin B12 deficiency, as a result of intrinsic factor deficiency. Many in the art believe that without performing a Schilling's test, intrinsic factor deficiency may not be proven, and intrinsic factor and parietal cell antibodies may be useful as surrogate markers of PA, with 73% sensitivity and 100% specificity. Lahner et al., “Pernicious anemia: New insights from a gastroenterological

point of view” World J Gastroenterol 15(41):5121-5128 (2009). PA is mainly considered a disease of the elderly, but younger patients represent about 15% of patients. PA patients may seek medical advice due to symptoms related to anemia, such as weakness and asthenia. Less commonly, the disease is suspected to be caused by dyspepsia. PA is frequently associated with autoimmune thyroid disease (40%) and other autoimmune disorders, such as diabetes mellitus (10%), as part of the autoimmune polyendocrine syndrome. PA is the end-stage of ABG. Longstanding Helicobacter pylori infection probably plays a role in many patients with PA, in whom the active infectious process has been gradually replaced by an autoimmune disease that terminates in a burned-out infection and the irreversible destruction of the gastric body mucosa. Human leukocyte antigen-DR genotypes suggest a role for genetic susceptibility in PA. PA patients are currently managed by cobalamin replacement treatment and monitoring for onset of iron deficiency. Moreover, PA may lead to possible gastrointestinal long-term consequences, such as gastric cancer and carcinoids.

### ATP4A Subdomain Constructs

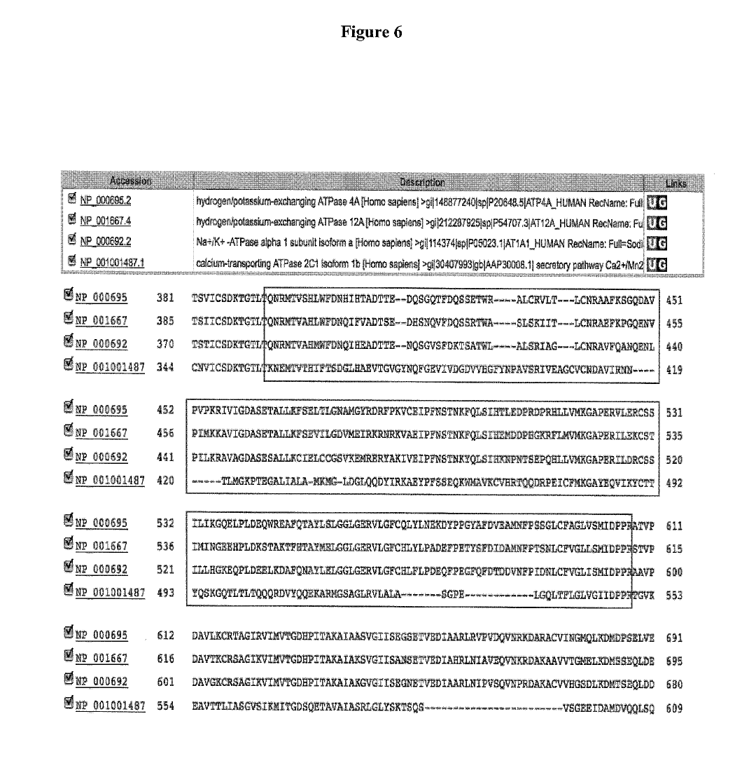

In one embodiment, the present invention contemplates a composition comprising an ATP4A homolog. See, FIG. 6. For example, ATP12A (i.e., H⁺/K⁺ transporting) an ATP4A homolog but, unlike ATP4A, ATP12A has a broad tissue distribution and may also be useful as a tumor biomarker. For example, high levels of ATP12A mRNA have been detected in colon and rectal cancers, thymus, pancreas, kidney, prostate, lung, and trachea. Other ATP4A homologous enzymes include, but are not limited to, ATP1A Na⁺/K⁺ ATPase and ATPase2C1 (Ca²⁺ transporting) that are found in most cell types.

In one embodiment, the present invention has identified a specific subdomain on ATP4A (i.e., for example, the D3.2 subdomain) that may be involved in the development of autoimmune diseases. This identification process began with evaluating full-length ATP4A enzymes that ultimately identified the D3.2 subdomain, as described below:

Step 1: ATP4A C-Term IVT IP Assays

A C-term ATP4A construct containing the final 252 amino acids was cloned from human stomach cDNA and tested in the RIA against sera newly diagnosed with T1D (e.g., new onset T1D sera). In most cases, new onset T1D sera was collected within six (6) months after T1D diagnosis.

Step 2: Full length ATP4B IVT IP Assays

Full-length ATP4B was cloned from human stomach cDNA and tested in the RIA against new onset T1D sera. An ATP4B C-term construct containing the terminal 225 amino acids was also engineered and tested against new onset T1D sera. Additionally, the full length ATP4B protein was glycosylated in vitro using pancreatic dog microsomes, and the modified protein was tested in the RIA against New Onset sera.

Step 3: ATP4A Assays with 3 Cytosolic Domains (D1-D3)

Using hydropathy analysis and the Uniprot database, the three largest cytoplasmic domains of the ATP4A protein were determined, and cloned from human stomach cDNA. Each probe was tested against new onset T1D sera

Step 4: ATP4A Subdomain Assays of D3

Using the crystal structure of a highly homologous Na“/K” transporter to examine potential conserved tertiary structure, the ATP4A D3 construct was cloned into two regions and tested in the RIA against new onset T1D sera.

Step 5: ATP4A D3.2 Subdomain Optimization

Boundaries of the ATP4A D3.2 subdomain probe were expanded and trimmed in 10-amino acid increments. A series of six constructs were cloned and tested in the RIA against new onset T1D sera with various permutations of N- and C-terminal boundaries.

In one embodiment, the present invention contemplates a method of detecting autoimmune antibodies comprising a radiochemical-based assay using an ATP4A D3.2 antigen capable of detecting 95% of diseased subjects with 100% specificity. In one embodiment, the ATP4A D3.2 antigen comprises amino acids (Q)₄₅-(P)247 of the ATP4A D3 subdomain ((T)1-(L) 434).

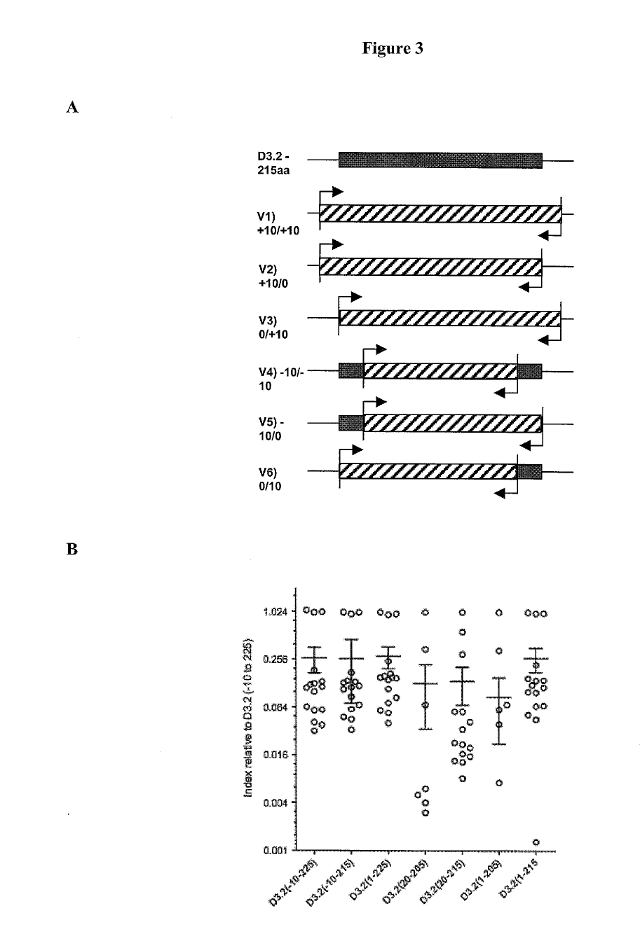

The data presented herein demonstrate that variants of the ATP4A D3.2 subdomain have differing capability to detect autoimmune antibodies. For example, D3.2 variants (V) having either extensions or deletions were constructed from the full length D3.2 subdomain. See, FIG. 3A. The sensitivity of each variant was compared against the full length 213(Q45-P247) amino acid subdomain. The V1 variant comprised a ten amino acid N-terminal extension and a ten amino acid C-terminal extension. The V2 variant comprised a ten amino acid N-terminal extension. The V3 variant comprises a ten amino acid C-terminal extension. The V4 variant comprised a ten amino acid N-terminal deletion and a ten amino acid C-terminal deletion. The V5 variant comprises a ten amino acid N-terminal deletion. The V6 variant comprised a ten amino acid C-terminal deletion.

Each variant was tested using sera collected from newly diagnosed diabetes type 1 patients (e.g., new onset T1D sera). See, FIG. 3. The constructs that have the best diagnostic performance were also observed to have the lowest background, thereby improving the specific signal. See, Table 2.

TABLE 2
ATP4A D3.2 Subdomain Variant Background Signal Comparison

| | (−)C | (+)C |
|---|---|---|
| D3.2 | 83.0 | X |
| V1 | 82.9 | 9636.5 |
| V2 | 92.3 | 9242 |
| V3 | 85.6 | 9332 |
| V4 | 439.3 | 455.5 |
| V5 | 132.4 | 1018 |
| V6 | 570.8 | 473 |

(−)C = Immunopreciptable counts in the absence of sera.
(+)C = Immnoprecipitable counts in the presence of sera.

The data demonstrate that variants V4, V5, and V6 showed the least capability of detecting autoimmune antibodies, presumably because these variants are lacking in either the N-terminal and/or C-terminal ends. On the contrary, the full length D3.2 and V1 demonstrated the optimal capability of detecting autoimmune antibodies (i.e., comprising at least 213 amino acids). Interestingly, a ten amino acid extension on both the N-terminal and C-terminal ends (i.e., for example, V1) demonstrated better detection than a single extension at either the N-terminal or C-terminal ends (i.e., for example, V2 and V3). Truncation of the C-terminal end (i.e., for example, V4 and V6) appears to increase non-specific binding substantially, whereas extensions of either the N-terminal or C-terminal ends (V1 and V2) has little effect on non-specific binding. Truncation of the N-terminal end (i.e., for example, V5) had little impact on non-specific binding but reduced total immunoprecipitable counts. The data suggest that variants comprising amino acids (Q)₄₅-(P)247 (D3.2) of subdomain D3 appear optimal (i.e., for example, V2). These types of refinement are impossible to perform with histochemical assays or ELISA assays based on native protein. The optimized function of variants comprising the first 213 amino acids of the ATP4A D3.2 subdomain is confirmed when using other variants as well. See, FIG. 3B.

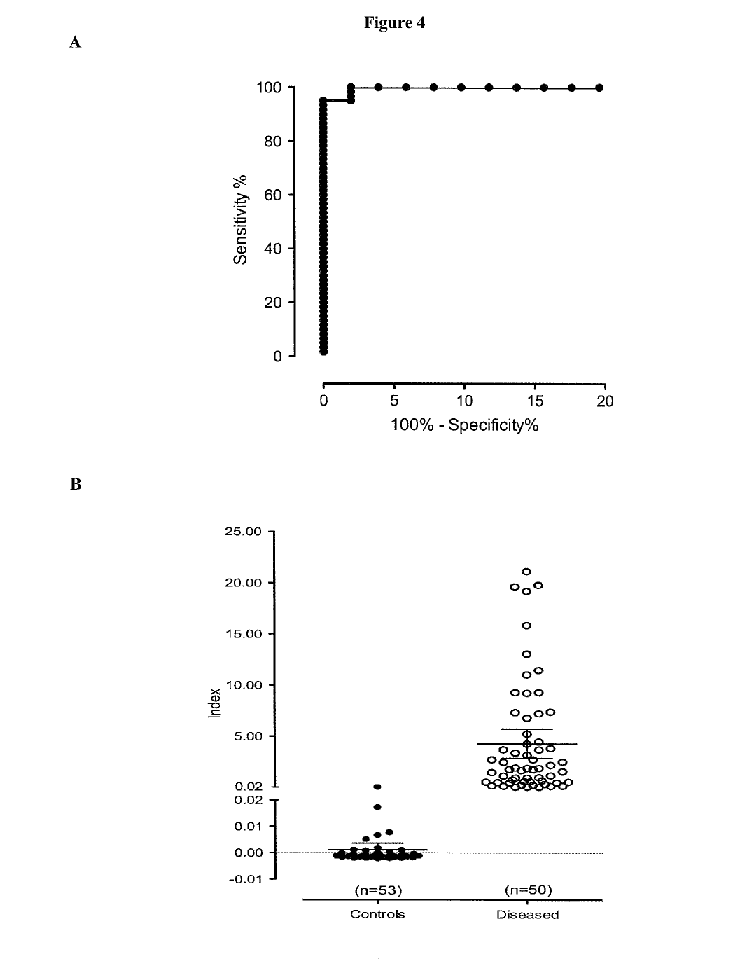

The above sera data was confirmed by comparison with a histological tissue biopsy study. Assays were performed on plasma from patients who had undergone optic biopsy to confirm a clinical ABG diagnosis. Sera from controls was assayed at 2.5 μl, ABG patients at 2.5 μl initially and repeated at 0.1 μl if index exceeded 0.8. The dilution was factored into the final calculation. The presence of plasma autoantibodies having ATP4A D3.2 epitopes demonstrated a 95-100% sensitivity in conjunction with a 80-100% specificity. See, FIGS. 4A and 4B.

### III. Autoimmune Disease Diagnostics

An ATP4A antigenic domain was tested against a panel of sera from new onset T1D patients which tested positive for the gold standard T1D autoantibodies (LAA, IA2A, GAD65A, and ZnT8A). The data presented herein show significant immunoreactivity to ATP4A (˜25%). Further, approximately 6% of first-degree relatives of T1D subjects who were sero-negative for T1D autoantigens were positive for ATP4A autoantibodies. The data also show that ATP4A antibody prevalence increases with the patient's age at onset of T1D, which is atypical of other T1D autoantibodies. Immunoreactivity to ATP4A, unlike that of T1D antigens, demonstrates a significant female gender bias in newly diagnosed T1D individuals.

In one embodiment, the present invention contemplates a method comprising an improved detection sensitivity for circulating autoantibodies that result in early pre-clinical diagnosis of autoimmune diseases (i.e., for example, autoimmune body gastritis and/or pernicious anemia) wherein a preclinical therapeutic intervention can be implemented. Such preclinical intervention prevents the development of secondary conditions including, but not limited, to diabetes mellitus and/or gastric carcinoma. Autoimmune diseases such as ABG are highly correlated with autoimmune disorders such as thyroiditis, type 1 diabetes, Addison's disease and vitiligo. In one embodiment, the present method contemplates a method for diagnosing autoimmune diseases including, but not limited to, autoimmune body gastritis (ABG), pernicious anemia, thyroiditis, type 1 diabetes, Addison's disease, or vitiligo. In one embodiment, the method further comprises detecting an autoantibody having affinity for a parietal cell ATP4 enzyme. In one embodiment, the autoantibody comprises affinity for the D3 domain of the ATP4A enzyme. In one embodiment, the D3 domain comprises the D3.2 subdomain.

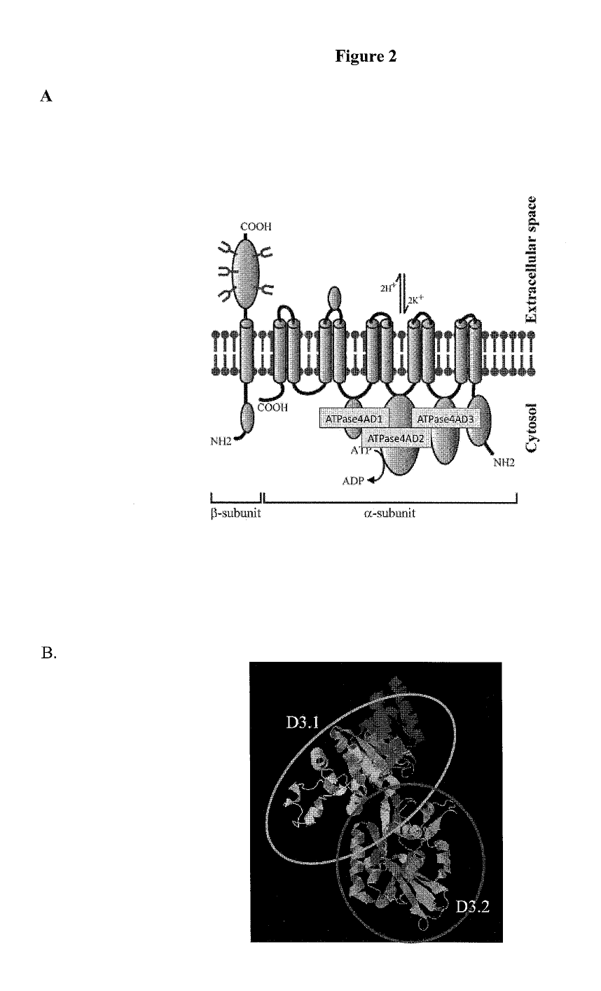

The presently disclosed assay was the result of an iterative process resulting in the identification of an optimized epitope that significantly improved the detection sensitivity of autoantibodies directed to ATP4 enzymes. The ATP4A subunit comprises several components having different relationships with the plasma membrane. It is believed that the H^{+/K}⁺-ATPase comprises an enzyme subunit structure similar to that reported for Na⁺/K⁺ ATPase. See, FIG. 2A. For example, an ATPase C-terminal end usually comprises primarily an intracellular portion. The ATPase N-terminal end may also comprise a cytosolic portion. Adjacent to the D1 domain (predicted to be a cytosolic loop) is an amino acid sequence denoted as a D2 domain, that is believed to comprise a phosphorylation-dephosphorylation domain. Then, between the D2 domain and the C-terminal transmembrane portion is located what is believed as the ionopore region, denoted as the D3 domain.

Although it is not necessary to understand the mechanism of an invention, it is believed that the above ATP4A D3.2 epitope assay comprises sufficient specificity and sensitivity within a patient population to diagnose many autoimmune diseases. For example, an ATP4A D3.2 epitope detection may suggest that some autoimmune diseases comprise an underlying defect in central immune tolerance that can be triggered by a environmental factor (i.e., for example, Helicobacter pylori infection). When autoantibody positive asymptomatic individuals are identified in early stages of an autoimmune disease, the initiation of conventional therapeutic intervention (i.e., for example, vitamin B12 supplementation) may be warranted even if a deficiency is not yet clinically apparent. Avoidance of vitamin B12 deficiency may have very important health consequences and would seem to be a very low risk intervention in terms of adverse outcomes.

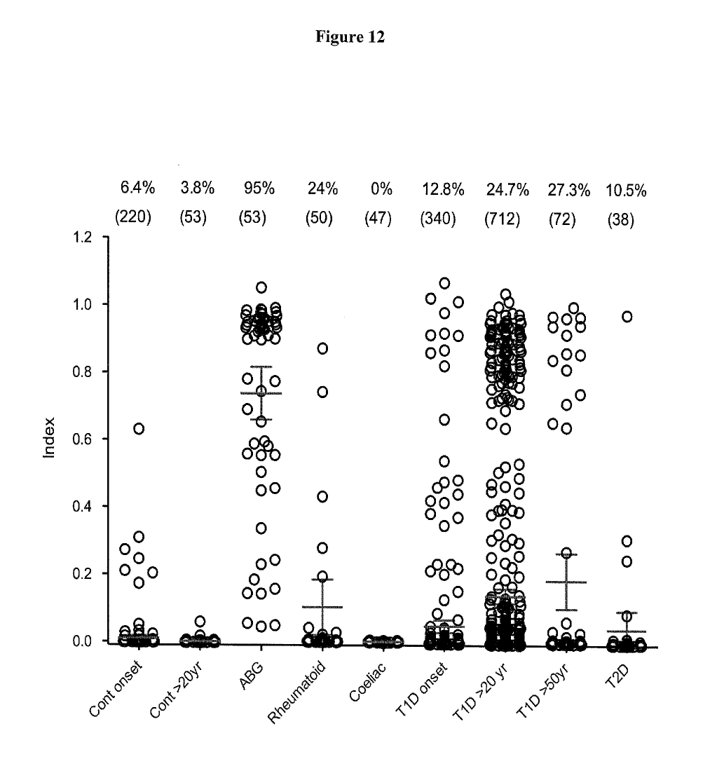

The data presented herein demonstrate that autoimmune antibodies associated with numerous autoimmune diseases may have, in common, an ATP4A D.3.2 epitope. For example, diseases including, but not limited to, type 1 diabetes, thyroditis (both Hashimoto's and Grave's Disease), rheumatoid arthritis, acute body gastritis, and/or pernicious anemia. It can be seen that differences between controls and diabetics in a new onset group is less marked than at later ages. See, FIG. 12. This may, in part, relate to the better HLA matching in the NO group (i.e., first degree relatives of the T1D subjects). There was an association with antibodies with DR3 and or DR4, but not DR5-I (data not shown).

A. Pernicious Anemia

While autoimmune body gastritis (ABG) and pernicious anemia are suspected to be related, ABG diagnosis is usually based on biopsy of inflammatory cells in the stomach. On the other hand, pernicious anemia has been associated with vitamin K deficiency. However, other conditions also present with vitamin B12 deficiency including, but not limited to, infection with the tapeworm Diphyllobothrium latum, gastric bypass surgery (i.e., for example, after a Roux-en-Y bypass) and chronic gastritis (i.e., for example, due to a Helicobacter pylori infection). While various forms of therapy involving dietary liver and/or liver extract treatments were common from the 1920's they were replaced with cobalamin treatment. Rickes et al., “Vitamin B12, A Cobalt Complex” Science 108(2797):134 (1948). Vitamin B12 deficiency is mediated by a reduction in IF, a product of the parietal cell, which is responsible for vitamin B12 binding and subsequent absorption. ABG can cause B12 deficiency either through the loss of the PC by autoimmunity or by intrinsic factor autoantibodies blocking the binding of cobalamin to IF and thus its uptake by the gut. ABG is also frequently associated with iron deficiency anemia by virtue of the need for gastric acidification to release iron from food in a readily absorbable form. Untreated, vitamin B12 deficiency is believed to result in pernicious anemia, and if untreated, irreversible neurological damage and death.

Symptoms of pernicious anemia may include, but are not limited to, anemia, fatigue, low blood pressure, rapid heart rate, pallor, shortness of breath, difficulty in proprioception, mild cognitive impairment, neuropathic pain, frequent diarrhea, paresthesias (i.e., for example, due to B12 deficiency affecting nerve function), jaundice (i.e., for example, due to impaired formation of blood cells), glossitis or swollen red tongue (i.e., for example, due to B-12 deficiency), presentation with hyperthyroidism or hypothyroidism, personality changes, or memory changes.

Another association between ABG and pernicious anemia may be related to a gastric membrane-bound proton pump, H⁺/K⁺-ATPase (ATP4). This proton pump may be localized in gastric parietal cells (PCs) and is believed responsible for maintaining stomach acidity. It is believed that H⁺/K⁺-ATPase may comprise an autoantigen, thereby generating an autoantibody that results in parietal cell loss and ABG development. This loss in parietal cells induces a compensatory increase in gastrin secretion that is believed to increase the risk of gastric carcinoma. In one embodiment, the ATP4 enzyme comprises a multi-spanning P2-type ATPase located in an apical plasma membrane. In one embodiment, the apical plasma membrane comprises a stomach parietal cell apical plasma membrane. In one embodiment, the ATP4 enzyme comprises at least two subunits. In one embodiment, a first subunit comprises an alpha (A) subunit. In one embodiment, the alpha subunit comprises a catalytic domain having an amino acid sequence of approximately 1033 amino acids. In one embodiment, the second subunit comprises a beta (β) subunit. In one embodiment, the beta subunit comprises a plurality of glycosylations located on an amino acid sequence of approximately 291 amino acids. Although it is not necessary to understand the mechanism of an invention, it is believed that ATP4 secretes H⁺ in exchange for K⁺ using ATP hydrolysis and can be a therapeutic target for drugs to control excess acid and dyspepsia and gastric ulcers. Alternative enzymes having functional similarity and/or sequence homology to ATP4A include but are not limited to ATP12A (i.e., for example, a potential biomarker for skin, kidney prostate and bowel tumors), ATP1 (i.e., for example, Na⁺/K⁺ ATPases), Ca type 2 ATPase, and Ca type 3 ATPase. Further, ABG autoantibodies are known to cross react with ATP12A, but only to a limited extent.

B. Diabetes

In one embodiment, the present invention contemplates a composition comprising an ATP4A homolog having specific reactivity with an antigen in T1 diabetic (T1D) sera.

The data presented herein demonstrate that the D3.2 subdomain of each ATP4A homolog in FIG. 6 was cloned and used in the presently disclosed radioimmunoprecipitation assay. The cloned D3.2 subdomains were tested with T1D sera that also demonstrated a high, medium or low titer to an ATP4A D3.2 subdomain antigen. See, Table 3.

### Tables 3 a and b: Comparative Reactivity Of ATP4A Homologs In T1D Sera

3a: Experimental (DM20+, trays 2, 3, 5, 8)

| ATP4A | High - 1 μl | | High - 0.1 μl | | Medium | | Low | |
|---|---|---|---|---|---|---|---|---|
| Homolog | 535381 | 546365 | 535381 | 546365 | 558826 | 562199 | 537181 | 546081 |
| 1A1 | 434 | 159 | 118 | 65 | 129 | 99 | 129 | 91 |
| 2C1 | 257 | 248 | 129 | 136 | 251 | 265 | 337 | 262 |
| 12A | 1754 | 241 | 412 | 87 | 227 | 177 | 118 | 117 |
| 4A | 12376 | 9763 | 12653 | 6295 | 12025 | 11275 | 2511 | 1864 |

Negative Control Data

| | 1A1 | 2C1 | 12A | 4A |
|---|---|---|---|---|
| (—)C | 107 | 289 | 138 | 191 |
| SD | 16 | 19 | 48 | 181 |
| C/O | 188 | 386 | 381 | 1097 |

3b: Additional Sera

| | Sample ID | | | | | | | | | |
|---|---|---|---|---|---|---|---|---|---|---|
| | 551066 | | 551107 | | 551775 | | 552047 | | 553287 | |
| | 2.5 μl | 1 μl | 2.5 μl | 1 μl | 2.5 μl | 1 μl | 2.5 μl | 1 μl | 2.5 μl | 1 μl |
| 1A1 | 119 | 91 | 128 | 93 | 115 | 100 | 155 | 158 | 95 | 91 |
| 12A | 86 | 70 | 1190 | 645 | 162 | 113 | 249 | 128 | 163 | 119 |
| 4A | 11291 | 11400 | 9335 | 11834 | 12283 | 12214 | 12192 | 11699 | 11915 | 11679 |

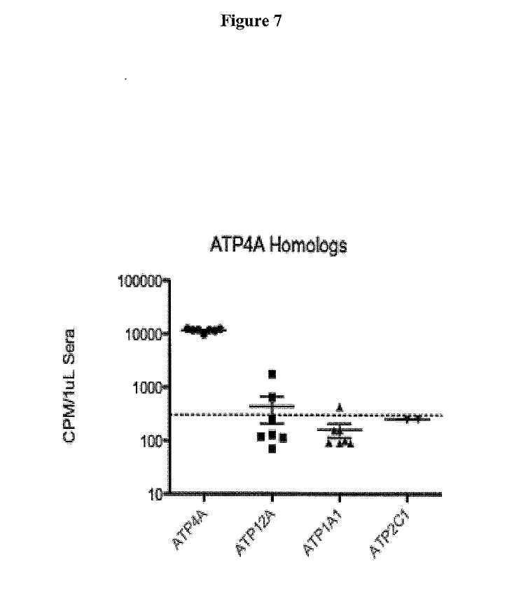

Reactivity to ATP4A (4A) is by far the highest, while ATP12A (12A) reactivity was evident in a subset of highly reactive ATP4A positive samples. While ATP12A reactivity was not proportional to ATP4A, the data suggest that ATP 12A might be an independent marker for other autoimmune diseases (i.e., for example, diabetes mellitus). For example, alignments of ATP4A and ATP12A, and comparisons of surface residues predicted by the crystal structure of ATP1A, has identified at least 8 amino acid residues that are uniquely shared between ATP4A and ATP12A but not the other homologs. See, FIG. 7. Although it is not necessary to understand the mechanism of an invention, it is believed that ATP4A and ATP12A may comprise a shared epitope.

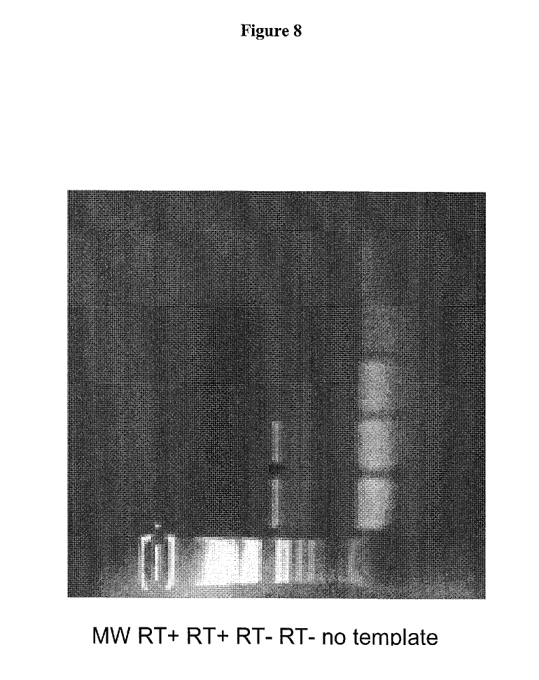

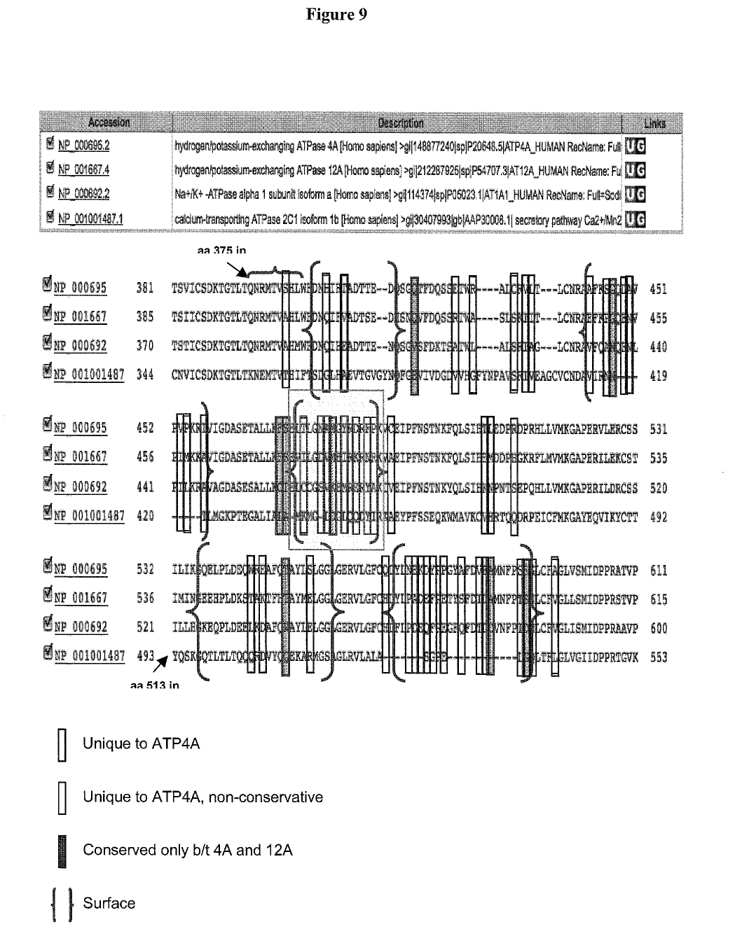

The above data suggested that there might be a pancreatic islet ATP4A homolog that might be a diabetes autoimmune target. Consequently, degenerate primers for a cDNA segment encompassing the D3.2 subdomain in the ATP1A, ATP4A, ATP12A and ATP2C1 homologs were used to amplify human pancreatic islet cDNA. The data show that when the 1200 bp of PCR amplified DNA was inserted into the pcDNA3.1 vector and 20 clones sequenced, only ATP1A1 (Na⁺/K⁺ transporter, alpha 1) polypeptide sequences were obtained. See, FIG. 8 (lanes 2 & 3). These data suggest that Type 1 diabetes (T1D) humoral autoreactivity to ATP4A may not be due to pancreatic islet ATP4A expression. One explanation of the above data is that ATP1A1 is much more abundant than a cross-reacting minor ATPase form. Identifying the cross-reacting minor ATPase form might be accomplished using a more exhaustive sequencing analysis. Alternatively, cross reactive pancreatic islet targets may be identified by preabsorption with recombinant ATP4A and other homologues followed by restriction enzyme PCR based cloning to eliminate the ATP1A1 product. However, when specific oligonucleotide primers designed to amplify subdomain ATP4A D1 were employed in PCR reactions using islet cDNA as template, 3 of 7 clones were identical to ATP4A (D1) implying the expression of ATP4A in the pancreatic islet. The evidence above does not rule out contaminating DNA sources and further analysis is required.

The presence of anti-parietal cell antibodies has previously been reported in other autoimmune disorders including thyroiditis, Addison's disease, and type 1 diabetes but T1D patients are potentially at higher risk of developing pernicious anemia, celiac disease, or Addison's disease. What was unclear in these previous studies was whether ATP4A/B or another antigen is the target. Although it is not necessary to understand the mechanism of an invention, it is believed that the presently disclosed immunoprecipitation assay has sufficient specificity that it would be unlikely to detect anything other than autoantibodies comprising an ATP4A D3.2 subdomain binding site.

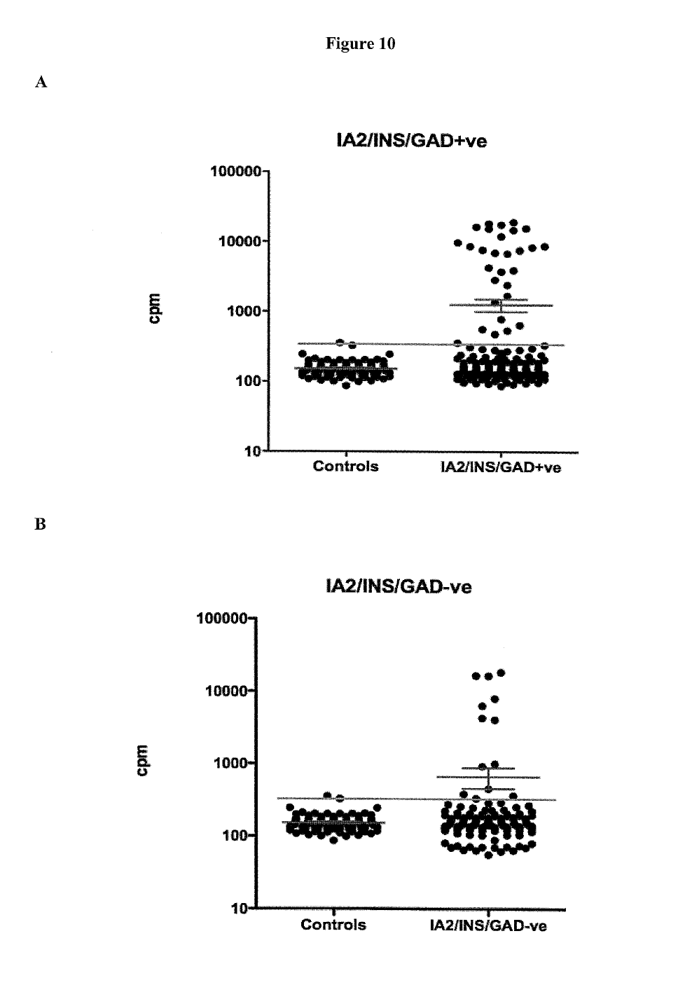

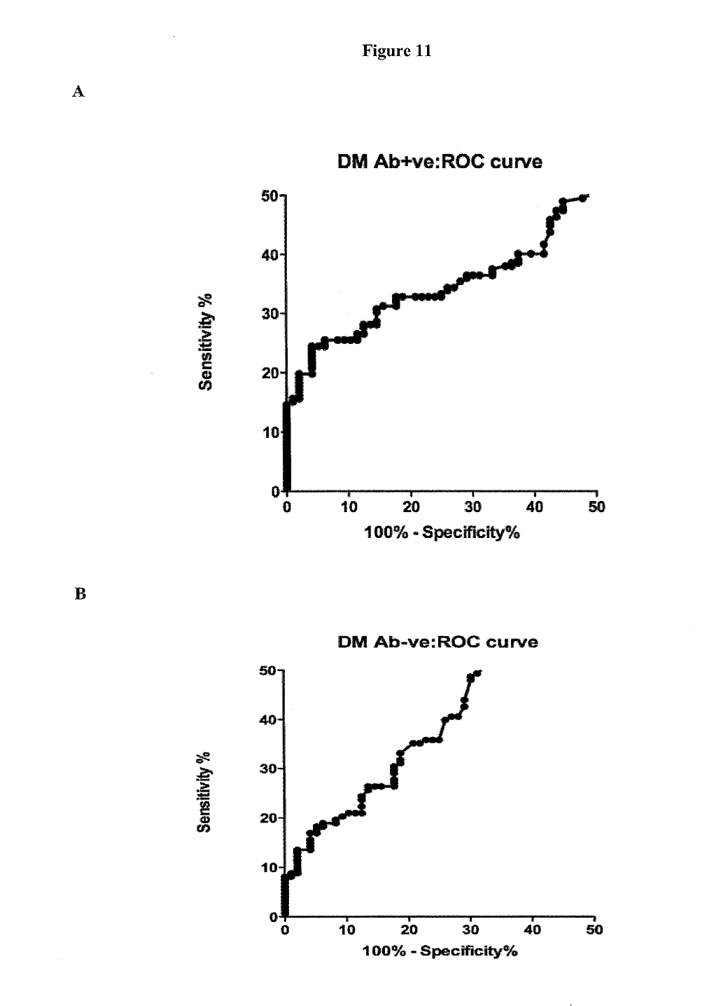

The ATP4A D3.2 subdomain immunoprecipitation assay was compared to data from newly presented diabetes mellitus (DM) patients identified with known T1D autoimmune antibodies (T1AA). These assays were performed with 2.5 μl serum mixed with 20,000 cpm of in vitro translated ATP4A D3 product. The data demonstrate a prevalence of autoimmune antibodies in non-diabetic controls around 6% at low titer, while the prevalence in new onset diabetes patients is around 18-25% at high titer. See, FIG. 10. These studies suggest that ATP4A D3.2 subdomain autoantibodies present in T1D are correlated with classic T1D autoimmune antibodies. The data show that autoantibodies comprising an ATP4A D3.2 subdomain binding site are associated with T1D at a sufficient sensitivity and selectivity to facilitate the diagnosis of diabetes. See, FIGS. 11A and 11B.

Further analysis of 135 T1D subjects assayed by ATP4A D.3.2 subdomain radioimmunoprecipitation demonstrated that levels of autoantibodies comprising ATP4A D3.2 subdomain binding sites increased in type 1 diabetes patients as age increased. See, FIG. 13. Further, analysis demonstrated that the prevalence of autoantibodies comprising ATP4A D3.2 subdomain binding sites was higher in diabetics (16%) than in age matched controls (8%) (data not shown).

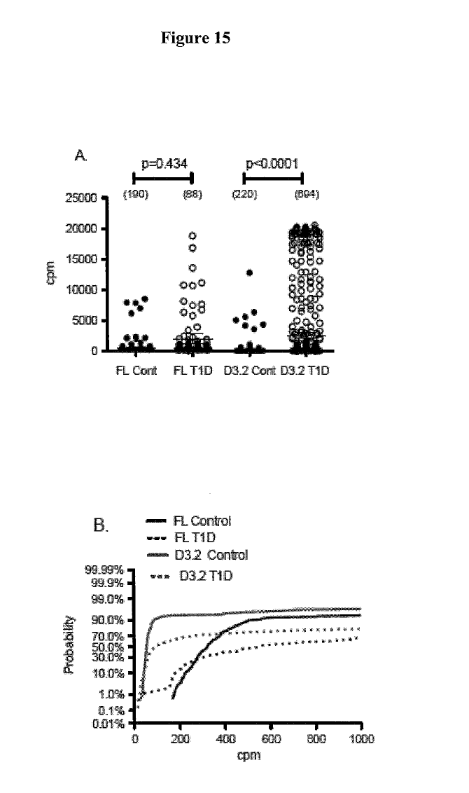

When immunoprecipitation detection of T1D autoantibodies using a full length (FL) ATP4A antigen was compared between control patients (i.e., not diagnosed with diabetes) and T1D patients, some controls tested positive. See, FIG. 15A. However, when the data was analyzed using Receiver Operator Curve statistical analysis, the T1D autoantibodies were detected with a sensitivity of up to 35% with a specificity of 95%, but only with an area under the curve calculation of 0.529 and a Mann-Whitney non-parametric statistic demonstrating statistical insignificance (p=0.438). It is clear, however, that the non-diabetic control population incorporates individuals who show autoreactivity to the ATP4A molecule (i.e., for example, ˜5-8%).

The detection of T1D autoantibodies using an ATP4A D3.2 epitope resulted in the proper diagnosis of diabetes with a 22% sensitivity at 95% specificity. In contrast to the FL ATP4A antigen, however, the D3.2 epitope data had an area under the curve of 0.780 and a Mann-Whitney statistical significance of <0.0001. See, FIG. 15A. Although it is not necessary to understand the mechanism of an invention, it is believed that the improved diagnosis of diabetes when using ATP4A D3.2 epitopes is also reflected in a frequency distribution analysis showing that the D3.2 construct was most likely more precise by virtue of having a lower control background plateau (i.e., 100 versus 500 cpm). See, FIG. 15B. Further, these data show a sharper discrimination between diabetic subjects that were antibody positive diabetic patients that were antibody negative.

While it is clear that there are non-diabetic individuals who test positive for ATP4A, it is believed that this observation is probably due binding to a different epitope than that recognized in the majority of diabetic patients. These data show that an empirical process is required to define a major diabetic epitope and tailor a specific detection assay in order to associate ATP4A reactivity with T1D.

C. Autoimmune Body Gastritis (ABG)

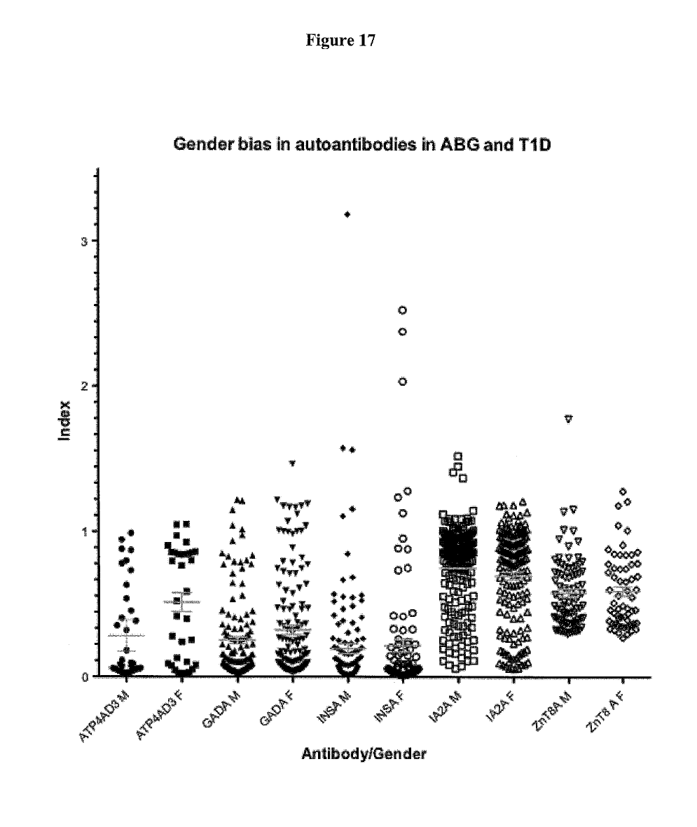

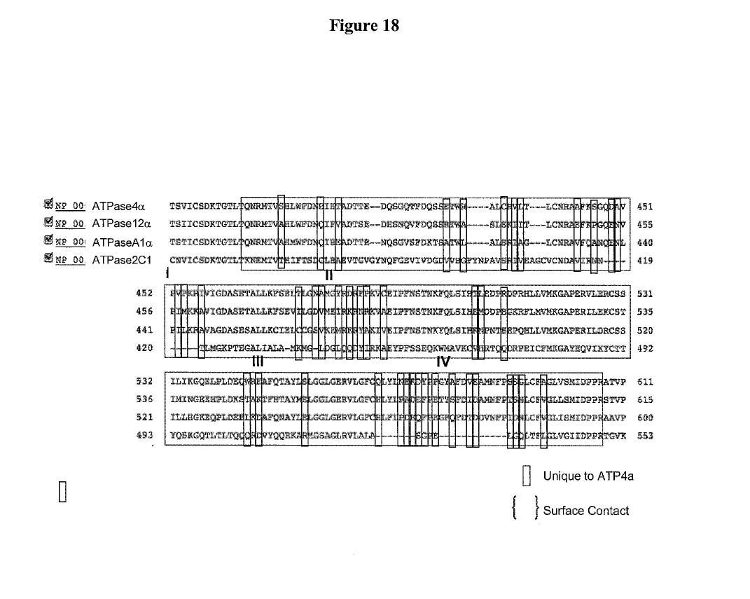

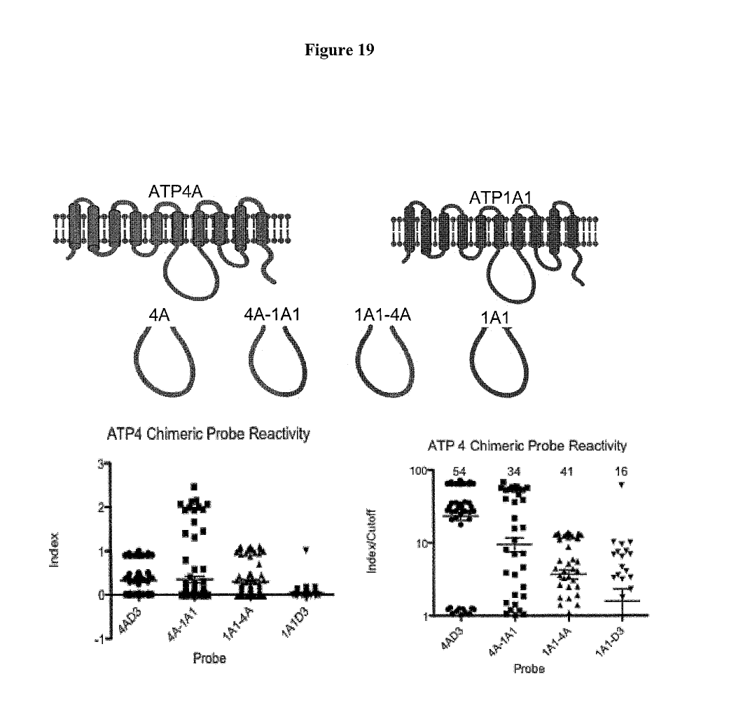

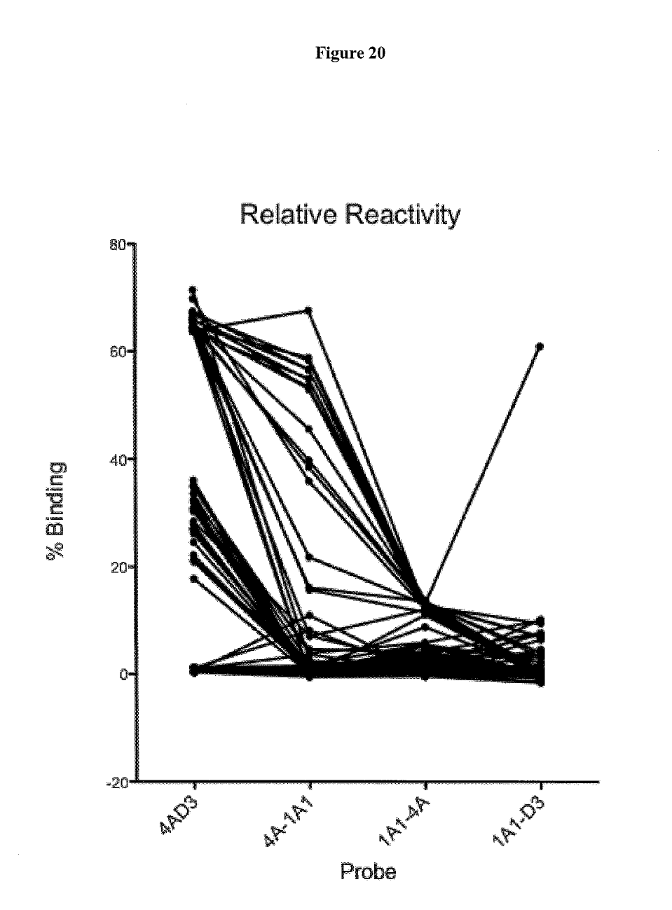

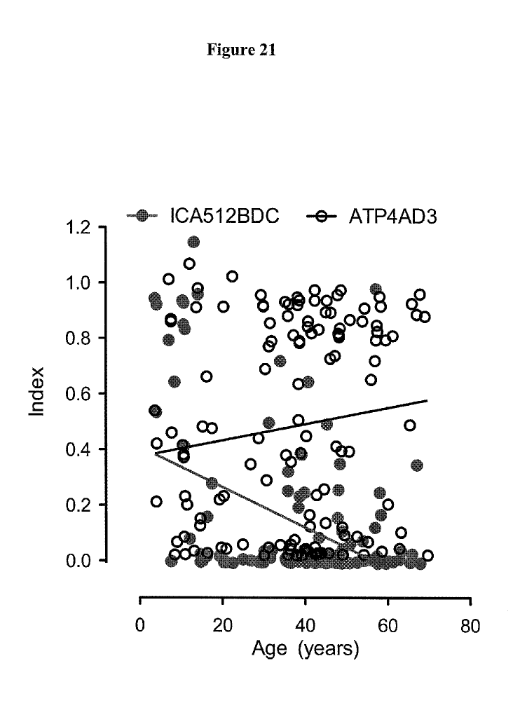

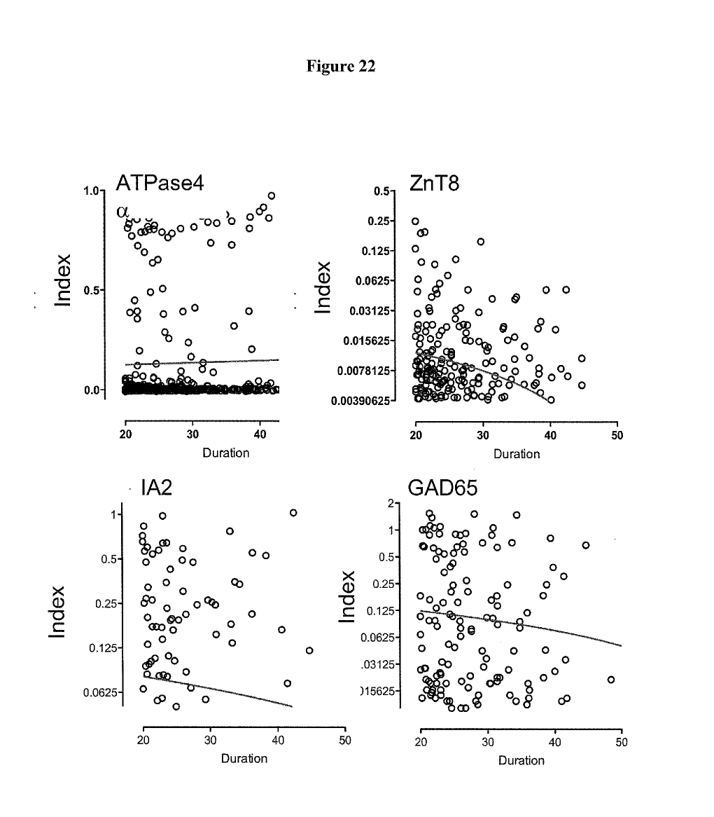

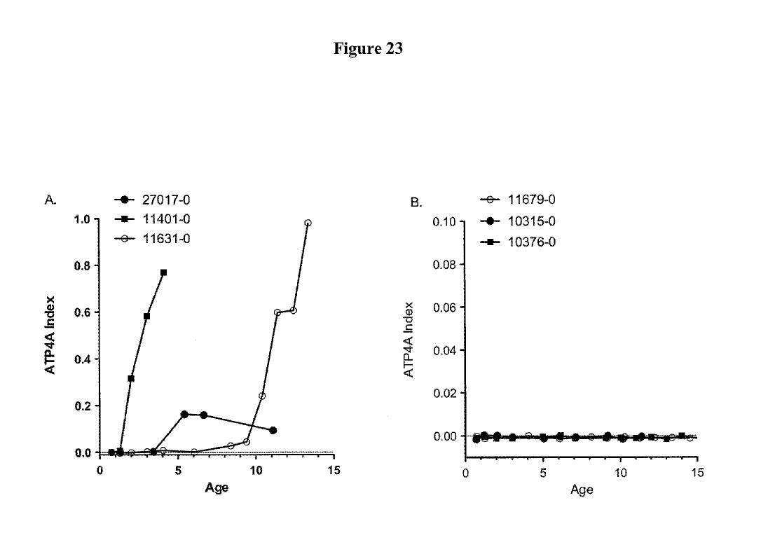

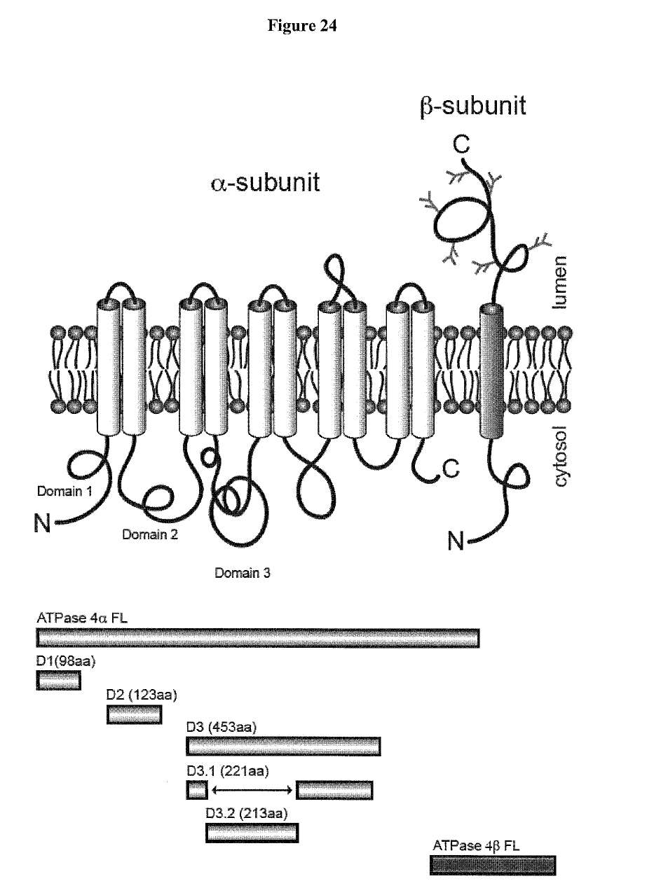

In one embodiment, the present invention contemplates an ATPase autoantibody assay that is molecularly optimized to target an ATP4A antigen that can be used to diagnose autoimmune body gastritis (ABG). The data presented herein demonstrate that a significant proportion of new onset T1D individuals exhibiting ABG symptoms harbor ATP4A autoantibodies. In one embodiment, an ATP4A autoantibody detected in an ABG patient comprises a very early biomarker for PA development. In one embodiment, the ATP4A autoantibody profile is distinctive as compared to previously used T1D autoantibodies including but not limited to GAD65, ICA512, MIAA, ZnT8, GADA, INSA, IA2A (e.g., “gold standard T1D autoantibodies). In one embodiment, the ATP4A autoantibody levels increase in proportion with the patient's age at T1D diagnosis. See, FIG. 21. In one embodiment, the ATP4A autoantibody index remains constant relative to the patient's age at T1D diagnosis. See, FIG. 22. In one embodiment, the ATP4A autoantibody index is higher in females that in males. See, FIG. 17. Although it is not necessary to understand the mechanism of an invention, it is believed that no gender bias exists for “gold standard” T1D autoantibodies measurements. In one embodiment, the detection of ABG-ATP4A autoantibodies provides an early ABG diagnosis. In one embodiment, the detection of ABG-ATP4A autoantibodies identifies a T1D-at risk patient subset who may benefit from simple, efficient, and cost-effective preventative therapies such as prophylactic vitamin B12. In one embodiment, the T1D at risk patient is a child. In one embodiment, the child is less than fifteen years of age. See, FIG. 23.

As discussed herein, a full length ATP4A gene has been cloned and a region of major immunoreactivity to optimize immune complex formation, while minimizing background reactivity, was empirically determined. For example, a major antigenic domain of ATP4A was subcloned into pcDNA3.1 (Invitrogen), a vector which has previously been employed for coupled in vitro transcription/translation reactions to generate ³⁵S-labelled antigen probes for use in radioimmunoprecipitation assays (RIAs). Wenzlau et al., “The cation efflux transporter ZnT8 (S1c30A8) is a major autoantigen in human type 1 diabetes” Proceedings of the National Academy of Sciences of the United States of America USA 104:17040-176045 (2007).

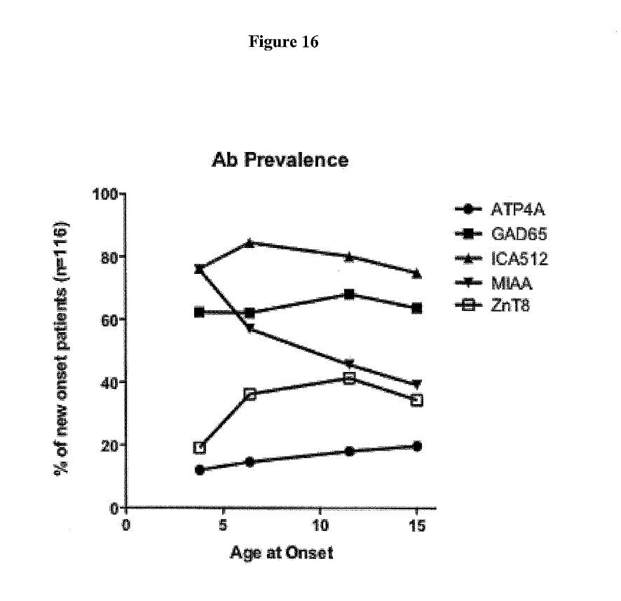

The data presented herein demonstrates the detection of ATP4A antibodies in a panel of 116 sera from new onset T1D subjects (i.e., for example, less than 6 months duration). See, Example III. These patients were followed prospectively and had been stratified on the basis of immunoreactivity to the T1D autoantibodies for insulin (INS, MIAA), the 65-kD form of glutamatic acid decarboxylase (GAD65), zinc transporter 8 (ZnT8) and insulinoma autoantigen-2 autoantibodies (IA2). See, FIG. 16. Individual autoantibodies demonstrated their characteristic age of onset prevalence profile. This persistence of GAD65 autoantibodies remained consistent independent of age of T1D onset while insulin autoantibodies (typically) declined. Measurement of IA2 (ICA512) autoantibodies likewise diminishes with advanced age of onset frequently in parallel (albeit not significant) with ZnT8 autoantibodies. In striking contrast, ATP4A autoantibodies do not mimic the established profiles for islet cell autoantibodies or those associated with other related autoimmune diseases such as Addison's and celiac disease (data not shown) but rather demonstrate a consistent increase with age of onset of T1D.

A second collection of sera derived from another cohort of newly diagnosed T1D individuals (<6 months, n=463) was assayed for the prevalence of autoantibodies against ATP4A, indicative of autoimmune gastritis, and INS, IA2, GAD65, and ZnT8 associated with T1D. See, FIG. 17. Twenty-five percent of sera from these patients demonstrated significant immunoreactivity to the ATP4A antigen (not shown). In contrast, RIAs conducted with sera obtained from first degree relatives of T1D individuals negative for the classic T1D autoantibodies, demonstrated 6% positivity for ATP4A autoantibodies (not shown).

Although it is not necessary to understand the mechanism of an invention, it is believed that that the presence of ATP4A autoantibodies may be due to a loss of immune tolerance that impacts more than one tissue (i.e., for example, gastric mucosa and/or pancreatic islets). For example, as ATP4A autoantibodies are found in a number of autoimmune disorders, the prevalence among first degree relatives may be a consequence of genetic predisposition linked to HLA and/or other interactions between specific at-risk genetic alleles or epigenetic factors linked to environmental agents.

When the indices for autoantibody positive samples were stratified according to gender, there was a significant gender bias in titers where the mean index for females and males is 0.5130 and 0.2820, respectively (p=0.0136; n=34 females, n=37 males). See, FIG. 17. There was no appreciable gender bias for any of the gold standard T1D autoantibodies: INS (p=0.8258) showing a mean index of 0.1940 for females (n=126) and 0.2164 for males (n=196), GAD (p=0.2865) with a mean index of 0.3238 for females (n=141) and 0.2508 for males (n=139), IA2A (p=0.2261) having a mean index of 0.6961 for females (n=161) and 0.7507 for males (n-182), and ZnT8 (p=0.8488) displaying a mean index of 0.5920 for females (n=60) and 0.5815 for males.

Children in the DAISY (Diabetes Autoimmunity Study in the Young) Study who were selected for high genetic risk of developing type 1 diabetes (first degree relative of T1D patient or DR3/4 DQ8 HLA genotype) have been followed prospectively for 15 years with annual serum samples measured for diabetes associated antibodies. See FIG. 23. These data show that approximately 5% developed T1D and 24% developed an autoimmune phenotype within 12 years after autoantibody detection. For example, ATP4A D3.2 antibodies were present in a higher proportion of islet antibody positive individuals (7-14% of cases n=149) than in islet antibody negative controls (2.43% n=206). Interestingly, as in the case of diabetic subjects ATP4A antibodies when present in the controls appeared at an early age and persisted with time. See, FIG. 23A. There was no evidence of transient ATP4A antibodies. See, FIG. 23B.

### IV. Kits

In another embodiment, the present invention contemplates kits for the practice of the methods of this invention. The kits preferably include one or more containers containing a labeled ATP4A D3.2 subdomain antigen. The kit can optionally include a nucleic acid sequence encoding an ATP4A D3.2 subdomain antigen. The kit can optionally include a plurality of buffers and reagents that are compatible with the ATP4A D3.2 antigen.

The kit can optionally include enzymes capable of performing PCR (i.e., for example, DNA polymerase, Taq polymerase and/or restriction enzymes). The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the above containers to perform immunoprecipitation techniques. The instructions may also provide a description of tagging an ATP4 D3.2 antigen with a radioactive label (i.e., for example, ³⁵S). In particular the disease can include any one or more of the disorders described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### V. Immunoprecipitation

Immunoprecipitation (IP) is a technique of precipitating a protein antigen out of solution using an antibody that specifically binds to that particular protein. This process can be used to isolate and concentrate a particular protein from a sample containing many thousands of different proteins. Immunoprecipitation is usually performed with an antibody coupled to a solid substrate at some point in the procedure. Other procedures also include precipitating the autoantibody with: i) another antibody or complexed to a bead; or ii) a physical precipitation of the antigen/antibody complex by a precipitating agent such as polyethylene glycol or ammonium sulfate.

Immunoprecipitation can be used to detect an antibody that specifically targets a single known protein. To facilitate identification of the antibody-protein complex, the protein may be tagged on either the C-terminal or N-terminal end of the protein of interest. The advantage here is that the same tag can be used time and again on many different proteins while screening different antibodies. Examples of tags may include, but are not limited to, the Green Fluorescent Protein (GFP) tag, Glutathione-S-transferase (GST) tag, the FLAG-tag tag, an enzyme such as horseradish peroxidase or β-galactosidase, a luciferase (firefly, Renilla or Glue), a chemiluminescent substrate, or a Europium complex. Alternatively, a protein may be tagged with a radioactive label (i.e., for example, ³⁵S, ³H, ¹⁴C, or ³²P).

Antibodies that are specific for a particular protein (or group of proteins) may be immobilized on a solid-phase substrate such as a superparamagnetic substrate or on an agarose substrate. The substrates with bound antibodies are then added to the protein mixture and the proteins that are targeted by the antibodies are captured onto the substrate via the antibodies (i.e., immunoprecipitated). Historically, a solid-phase support for immunoprecipitation has preferably been highly porous agarose substrates (i.e., for example, agarose resins or slurries). The advantage with this technology is a very high potential binding capacity as virtually the entire sponge-like structure of the agarose particle is available for binding antibodies which will in turn bind the target proteins. This advantage of extremely high binding capacity must be balanced with the quantity of antibody expected to contact the agarose beads. For example, one may calculate backward from the amount of protein that needs to be captured, to amount of antibody that is required to bind that quantity of protein, and back still further to the quantity of agarose that is needed to bind that particular quantity of antibody. The portion of the binding capacity of the agarose beads that is not coated with antibody will then participate in non-specific binding events. This elevates the level of random non-specifically bound proteins to the substrate which results in an increase in background signal that can make it more difficult to interpret results. For these reasons it is prudent to match the quantity of agarose (in terms of binding capacity) to the quantity of antibody that one wishes to be bound for the immunoprecipitation.

Alternatively, in contrast to the direct binding methods described above (which have an inherent disadvantage of requiring the tedious procedure of coupling each and every sample to a solid substrate) indirect binding assays may also be performed where an antibody complex is formed in solution with a labeled known antigen in the presence of an unknown amount antibody (i.e., for example, an autoantibody). The antigen/antibody binding complex may then be recovered by precipitating the solution with an agent such as protein A agarose or an antibody that recognizes all human immunoglobulins tethered to a support.

Once a solid substrate has been chosen, antibodies can be coupled to the substrate by, for, example, contacting the substrate with a biological sample. Next, the antibody-coated-substrate can be contacted with a labeled protein sample (i.e., for example, a labeled antigen comprising a protein epitope). At this point, antibodies that are stuck to the substrate will bind the labeled proteins for which they have specific affinity thereby completing the immunoprecipitation step. Next, the substrate is washed such that only the bound antibody-protein complex remains.

With an agarose substrate the washing steps may be accompanied by pelleting the agarose from the residual sample by briefly spinning in a centrifuge with forces between 600-3,000×g (times the standard gravitational force). This step may be performed in a standard microcentrifuge tube, but for faster separations, greater consistency and higher recoveries, the process is often performed in small spin columns with a pore size that allows liquid, but not agarose beads to pass through. After centrifugation, the agarose substrate may form a very loose fluffy pellet at the bottom of the tube.

Following the initial capture of a protein or protein complex, the solid support may be washed several times to remove any proteins not specifically and tightly bound to the support through the antibody. After washing, the precipitated protein(s) may be eluted and analyzed using scintillation counting, gel electrophoresis, mass spectrometry, western blotting, or any number of other methods for identifying constituents in the complex. Alternatively, filter plates configured in a 96-well format sequentially washed and drained with a vacuum apparatus may be employed for processing large numbers of samples. Bound signal may then be quantified directly on the filter plate with the appropriate detection instrument.

### VI. Detection Methodologies

A Detection of Nucleic Acids

mRNA expression may be measured by any suitable method, including but not limited to, those disclosed below.

In some embodiments, RNA is detected by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to an oligonucleotide probe. A variety of hybridization assays using a variety of technologies for hybridization and detection are available. For example, in some embodiments, TaqMan assays (PE Biosystems, Foster City, Calif.; See e.g., U.S. Pat. Nos. 5,962,233 and 5,538,848, each of which is herein incorporated by reference) are utilized. The assay is performed during a PCR reaction that exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR(RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

The method most commonly used as the basis for nucleic acid sequencing, or for identifying a target base, is the enzymatic chain-termination method of Sanger. Traditionally, such methods relied on gel electrophoresis to resolve nucleic acid fragments differing in size by one base pair wherein nucleic acid fragments are produced from a larger nucleic acid segment as a template. However, in recent years various sequencing technologies have evolved which rely on a range of different detection strategies, such as mass spectrometry and array technologies.

One class of sequencing methods assuming importance in the art are those which rely upon the detection of PPi release as the detection strategy. It has been found that such methods lend themselves admirably to large scale genomic projects or clinical sequencing or screening, where relatively cost-effective units with high throughput are needed.

Methods of sequencing based on the concept of detecting inorganic pyrophosphate (PPi), which is released during a polymerase reaction, have been described in the literature—for example (WO 93/23564, WO 89/09283, WO98/13523 and WO 98/28440). As each nucleotide is added to a growing nucleic acid strand during a polymerase reaction, a pyrophosphate molecule is released. It has been found that pyrophosphate released under these conditions can readily be detected, for example, enzymatically e.g. by the generation of light in the luciferase-luciferin reaction. Such methods enable a base to be identified in a target position and DNA to be sequenced simply and rapidly whilst avoiding the need for electrophoresis and the use of labels.

At its most basic, a PPi-based sequencing reaction involves simply carrying out a primer-directed polymerase extension reaction, and detecting whether or not that nucleotide has been incorporated by detecting whether or not PPi has been released. Conveniently, this detection of PPi-release may be achieved enzymatically, and most conveniently by means of a luciferase-based light detection reaction termed ELIDA (see further below).

It has been found that dATP added as a nucleotide for incorporation, interferes with the luciferase reaction used for PPi detection. Accordingly, a major improvement to the basic PPi-based sequencing method has been to use, in place of dATP, a dATP analogue (specifically dATPa) which is incapable of acting as a substrate for luciferase, but which is nonetheless capable of being incorporated into a nucleotide chain by a polymerase enzyme (WO98/13523).

Further improvements to the basic PPi-based sequencing technique include the use of a nucleotide degrading enzyme such as apyrase during the polymerase step, so that unincorporated nucleotides are degraded, as described in WO 98/28440, and the use of a single-stranded nucleic acid binding protein in the reaction mixture after annealing of the primers to the template, which has been found to have a beneficial effect in reducing the number of false signals, as described in WO00/43540.

B. Detection of Protein

In other embodiments, gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by identifying a label on the primary antibody. In another embodiment, the primary antibody is detected by monitoring binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

C. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personnel and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

D. Detection Kits

In other embodiments, the present invention provides kits for the detection and characterization of proteins and/or nucleic acids. In some embodiments, the kits contain antibodies specific for a protein expressed from a gene of interest, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

### EXPERIMENTAL

### Example I

### Immunoprecipitation: Basic Agarose Technique

1. Lyse cells and prepare a biological sample.
2. Attach antibody to agarose by contacting with a biological sample.
3. Incubate solution with antibody against a protein of interest (i.e., for example, an ATP4A D3.2 antigen).
4. Precipitate the complex of interest by adding Protein A thereby removing it from bulk solution.
5. Wash precipitated complex several times. Centrifuge each time between washes and then remove supernatant. After final wash, remove as much supernatant as possible.
6. Elute proteins from solid support (i.e., for example, by using low-pH or SDS sample loading buffer).
7. Analyze complexes or antigens of interest. This can be done in a variety of ways:
   a. Quantitating a radioactive label using a scintillation counter.
   b. SDS-PAGE (sodium dodecyl sulfate-polyacrylamide gel electrophoresis) followed by gel staining.
   c. SDS-PAGE followed by: staining the gel, cutting out individual stained protein bands, and sequencing the proteins in the bands by MALDI-Mass Spectrometry
   d. Transfer and Western Blot using another antibody for proteins that were interacting with the antigen followed by chemiluminescent visualization.

### Example II

### Commercial ELISA Versus ATP4A D3 Radiochemical/Immunoprecipitation Assay

Sera from 94 ABG patients were assayed by conventional ELISA and the ATP4A D3 radioimmunoprecipitation assay described above. An excellent concordance was observed for high titer samples but the ATP4A D3 radioimmunoprecipitation assay was more sensitive than the conventional ELISA for low and moderate titer samples (46 vs 13). See, FIG. 5. Further, the ELISA method showed 7 false positives.

### Example III

### ATP4 Radioimmunoassay

Serum samples were acquired after informed consent from patients, relatives and controls attending The Barbara Davis Center in compliance with IRB-approved protocols. Radioimmunoprecipitation assays (RIAs) were performed according to published procedures using ATP4A derivative antigen probes. Wenzlau et al., “The cation efflux transporter ZnT8 (S1c30A8) is a major autoantigen in human type 1 diabetes” Proceedings of the National Academy of Sciences of the United States of America USA 104:17040-17045 (2007).

Assays were conducted with 16 matched control samples and a pool of human sera with high-titer ATP4A antibodies. Cut-off indices were determined by the mean+/−5 SD of the intra-assay control values. The immunoprecipitation index was calculated by: sample−negative control mean/positive control mean−negative control mean.

We claim:

1. A complex comprising an autoimmune antibody having an ATP4A D3.2 subdomain binding site and an ATP4A D3.2 subdomain antigen attached to said ATP4A D3.2 subdomain binding site.

2. The complex of claim 1, wherein said autoimmune antibody is a stomach parietal cell antibody.

3. The complex of claim 1, wherein said autoimmune antibody is a pancreatic islet cell antibody.

4. The complex of claim 1, wherein said autoimmune antibody is a thyroid antibody.

5. The complex of claim 1, wherein said autoimmune antibody is a rheumatoid arthritis antibody.

6. The complex of claim 1, wherein said antigen comprises a label.

7. The complex of claim 6, wherein said label is a radioactive label.

8. The complex of claim 7, wherein said radioactive label is ³⁵S.

9. The complex of claim 1, wherein said antigen comprises an amino acid sequence.

10. The complex of claim 9, wherein said amino acid sequence comprises at least 215 amino acids.

11. A method, comprising:
a) providing;
i) a biological sample comprising an autoimmune antibody, wherein said autoimmune antibody comprises an ATPA D3.2 subdomain binding site;
ii) an ATP4A D3.2 subdomain antigen having specific affinity for said ATP4A D3.2 subdomain binding site; and
b) contacting said biological sample with said antigen under conditions such that said autoimmune antibody is identified.

12. The method of claim 11, wherein said conditions comprise immunoprecipitation of said autoimmune antibody.

13. The method of claim 11, wherein said conditions comprise identifying said autoimmune antibody at least at a 95% sensitivity.

14. The method of claim 11, wherein said conditions comprise identifying said autoimmune antibody at least at a 96% sensitivity.

15. The method of claim 11, wherein said conditions comprise identifying said autoimmune antibody at least at 97% sensitivity.

16. The method of claim 11, wherein said biological sample is a blood sample.

17. The method of claim 16, wherein said blood sample is selected from the group consisting of a whole blood sample, a plasma sample, and a serum sample.

18. The method of claim 11, wherein said biological sample is a stomach sample.

19. The method of claim 11, wherein said biological sample is a pancreas sample.

20. The method of claim 11, wherein said biological sample is a thyroid sample.

21. A method, comprising:
a) providing;
i) a patient exhibiting symptoms of an autoimmune disease;
ii) a labeled ATP4A D3.2 subdomain antigen; and
b) obtaining a biological sample from said patient; and
c) using said antigen to identify an autoimmune antibody in said biological sample.

22. The method of claim 21, said autoimmune antibody comprises an ATP4A D3.2 subdomain binding site.

23. The method of claim 21, wherein said autoimmune antibody is an autoimmune body gastritis antibody.

24. The method of claim 21, wherein said autoimmune antibody is a type 1 diabetes antibody.

25. The method of claim 21, wherein said autoimmune antibody is a pernicious anemia antibody.

26. The method of claim 21, wherein said autoimmune antibody is a thyroiditis antibody.

27. The method of claim 21, wherein said autoimmune antibody is an Addison's disease antibody.

28. The method of claim 21, wherein said autoimmune antibody is a rheumatoid arthritis antibody.

29. A method, comprising:
a) providing;
i) a patient exhibiting symptoms of an autoimmune disease;
ii) a labeled ATP4A D3.2 subdomain antigen; and
b) obtaining a biological sample from said patient; and
c) using said antigen to diagnose said autoimmune disease of said patient.

30. The method of claim 29, wherein said diagnosis is autoimmune body gastritis.

31. The method of claim 29, wherein said diagnosis is type 1 diabetes.

32. The method of claim 29, wherein said diagnosis is pernicious anemia.

33. The method of claim 29, wherein said diagnosis is thyroiditis

34. The method of claim 29, wherein said diagnosis is Addison's disease.

35. The method of claim 29, wherein said diagnosis is rheumatoid arthritis.

36. A method, comprising:
a) providing;
i) a patient at risk of developing symptoms of an autoimmune disease;
ii) a labeled ATP4A D3.2 subdomain antigen; and
b) obtaining a biological sample from said patient; and
c) using said antigen to identify an autoimmune antibody, wherein said autoimmune antibody comprises an ATP4A D3.2 subdomain binding site.

37. The method of claim 36, wherein said method further comprises after step (c), administering a therapeutic intervention.

38. The method of claim 37, wherein said therapeutic intervention comprises vitamin B12.

39. The method of claim 37, wherein said therapeutic intervention comprises an anticancer agent.

40. The method of claim 37, wherein said therapeutic intervention comprises an antidiabetic agent.

41. The method of claim 37, wherein said therapeutic intervention comprises an anti-gastrin agent.

42. The method of claim 37, wherein said therapeutic intervention comprises an anti-inflammatory agent.

43. The method of claim 36, wherein said conditions comprise immunoprecipitation of said autoimmune antibody.

44. The method of claim 36, wherein said conditions comprise identifying said autoimmune antibody at least at 95% sensitivity.

45. The method of claim 36, wherein said conditions comprise identifying said autoimmune antibody at least at 96% sensitivity.

46. The method of claim 36, wherein said conditions comprise identifying said autoimmune antibody at least at 97% sensitivity.

47. The method of claim 36, wherein said biological sample is a blood sample.

48. The method of claim 47, wherein said blood sample is selected from the group consisting of a whole blood sample, a plasma sample, and a serum sample.

49. The method of claim 36, wherein said biological sample is a stomach sample.

50. The method of claim 36, wherein said biological sample is a pancreas sample.

51. The method of claim 36, wherein said biological sample is a thyroid sample.

52. A method comprising:
a) providing;
i) a patient suspected of comprising an ATP4A autoantibody;
ii) a biological sample derived from said patient; and
iii) a labeled ATP4A antigen capable of binding to said ATP4A autoantibody;
b) contacting said labeled ATP4A antigen with said biological sample; and
c) determining an ATP4A autoantibody level.

53. The method of claim 52, wherein said ATP4A autoantibody comprises an ATP4A D3.2 subdomain.

54. The method of claim 52, wherein said patient is diagnosed with type 1 diabetes within the last six months.

55. The method of claim 52, wherein said patient is diagnosed as at risk for type 1 diabetes.

56. The method of claim 52, wherein said patient is diagnosed with autoimmune body gastritis.

57. The method of claim 56, said detection of said ATP4 autoantibody diagnoses autoimmune body gastritis.

58. The method of claim 52, wherein said ATP4A autoantibody level increases with the age of said patient.

59. The method of claim 52, wherein said method further comprises determining an ATP4 autoantibody index.

60. The method of claim 59, wherein said ATP4 autoantibody index is gender biased.

61. The method of claim 60, wherein said gender bias is female.

62. The method of claim 52, wherein said biological sample comprises a saliva sample.

63. The method of claim 52, wherein said biological sample comprises a blood sample.

64. The method of claim 63, wherein said blood sample is selected from the group consisting of a whole blood sample, a serum sample, and/ a plasma sample.

65. The method of claim 52, wherein said biological sample is a tissue sample.

66. A kit comprising:
a) a first container comprising a labeled ATP4A D3.2 subdomain antigen;
b) a second container comprising buffers and reagents compatible with said antigen; and
c) instructions describing how to use said first and second containers to identify an autoimmune antibody from a biological sample.
